(19) United States
(12) Patent Application Publication
(10) Pub. No.: US 2015/0018411 A1
(43) Pub. Date: Jan. 15, 2015

(54) COMPOUNDS AS PPAR BETA/DELTA INHIBITORS FOR TREATING PPAR BETA/DELTA-MEDIATED DISEASES

(71) Applicants: Wibke Diederich, Marburg (DE); Stefanie Dorr, Marburg (DE); Kerstin Kaddatz, Marburg (DE); Gerhard Klebe, Marburg (DE); Rolf Muller, Marburg (DE); Simone Naruhn, Ebsdorfergrund (DE); Veronika Pape, Unna (DE); Josefine Stockert, Marburg (DE); Philipp Manuel Toth, Marburg (DE)

(72) Inventors: Wibke Diederich, Marburg (DE); Stefanie Dorr, Marburg (DE); Kerstin Kaddatz, Marburg (DE); Gerhard Klebe, Marburg (DE); Rolf Muller, Marburg (DE); Simone Naruhn, Ebsdorfergrund (DE); Veronika Pape, Unna (DE); Josefine Stockert, Marburg (DE); Philipp Manuel Toth, Marburg (DE)

(73) Assignee: PHILIPPS-UNIVERSITAT MARBURG, Marburg (DE)

(21) Appl. No.: 14/123,815

(22) PCT Filed: Jun. 6, 2012
(86) PCT No.: PCT/EP2012/060777

§ 371 Date: Dec. 4, 2013

(30) Foreign Application Priority Data
Jun. 6, 2011 (DE) 102011104267.2

(51) Int. Cl. C07D 333/40; C07C 213/08

(52) U.S. Cl. CPC C07D 333/40; C07C 213/08
USPC 514/445; 549/64

(57) ABSTRACT

The present invention concerns substances which act as selective ligands of nuclear receptors of the PPAR beta/delta subtype and which can be used for the treatment of PPAR beta/delta-mediated diseases. The substances of this invention act as inhibitors of PPAR beta/delta receptors.

The present invention concerns substances which act as selective ligands of nuclear receptors of the PPAR beta/delta subtype and are thus suitable to be used for the treatment of PPAR beta/delta-mediated diseases. Substances of the present invention act as inhibitors of the PPAR beta/delta receptor.

Peroxisome-proliferator activated receptors (PPARs) are nuclear receptors which act as ligand-inducible transcription factors. The three known PPAR subtypes PPAR alpha, PPAR beta/delta and PPAR gamma thereby form heterodimers with members of the retinoid X receptor (RXR) family which subsequently bind to PPAR response elements (PPRE) on the DNA and regulate the activity of their target genes. PPARs act as sensors for fatty acids and eicosanoid metabolites which—like for example certain prostaglandins, leukotrienes or hydroxyeicosatetraenoic acids—have a function in immune regulation. This feature confers a central role on PPAR receptors in the fatty acid metabolism and for inflammatory processes. Consequently, PPAR receptors also play an important role for diseases like for example hyperlipidemia, diabetes, fibrosis, inflammatory diseases and cancer. To the inflammatory diseases belong among others Alzheimer's disease, arthritis, asthma, atherosclerosis, Crohn's disease, colitis, dermatitis, diverticulitis, hepatitis, irritable bowel syndrome, lupus erythematosus, nephritis, Parkinson's disease and ulcerative colitis.

Receptors of the PPAR beta/delta subtype have essential functions in the lipid- and glucose metabolism as well as in other disease-related biological processes like for example cell differentiation, proliferation, apoptosis and immune regulation. Furthermore, PPAR beta/delta also plays a role in tumorigenesis. The participation of PPAR beta/delta receptors in inflammation-associated processes is in addition reflected by various functions.

Endogenous ligands for PPAR beta/delta receptors are fatty acids like arachidonic acid and metabolites thereof like 15-hydroxyeicosatetraenoic acid (15-HETE) and prostaglandin I2 (PGI2, prostacyclin). In the absence of a ligand, PPAR beta/delta is often complexed with corepressors like SMRT or SHARP (“SMRT/HDAC I-associated repressor protein”). Substances acting as agonists of PPAR beta/delta receptors induce a conformational change of PPAR beta/delta which results in a dissociation of corepressors like for example SMRT and/or an interaction with specific co-activators like for example histone acetylases, with subsequent transcriptional activation of genes.

Furthermore, PPAR beta/delta is in addition able to regulate genes also independently of DNA binding. In the absence of a ligand, PPAR beta/delta interacts for example with BCL6 in macrophages and suppresses the repression of (pro-) inflammatory genes by BCL6 such as for example by mcp1, IL1b and mmp9. PPAR beta/delta also plays a key role for the differentiation, polarization and/or function of specific immune cells like for example macrophages and T-helper cells and is associated with pro-inflammatory mechanisms involved in psoriasis.

PPAR beta/delta agonists modulate among others also the effect of TGF-beta (transforming growth factor beta) and are thus able to contribute to the repression of genes with functions in immune regulation. TGF-beta is furthermore a frequently occurring cytokine in tumors. TGF beta-mediated SMAD proteins induce among others the transcription of the gene for angiopoietin-like protein ANGPTL4 which is, in addition to its function in the regulation the lipid metabolism, probably also involved in angiogenesis and tumor progression. It is common knowledge that the expression of the ANGPTL4 gene is also regulated by PPAR receptors.

The state of the art knows substances which act as specific, high-affinity bio-available agonists for the beta/delta subtype of PPAR receptors like GW501516, L165041, cPGI (carba prostacyclin) and GW2433. Of clinical relevance is primarily GW501516 which is already applied in a clinical trial (phase II) for the treatment of dyslipidemias (GlaxoSmithKline, study number: NCT00158899). However, no specific high-affinity inhibitory substances are known to date which allow a use as bio-available and reversible or competitive antagonists or as inverse agonists for PPAR beta/delta.

US 2004/0180892 describes sulfonamides which act as urotensin-II or CCR-9 antagonists. DE 60215145 T2 discloses sulfonamides which also act as urotensin-II antagonists.

The state of the art furthermore knows antagonists for the alpha or gamma subtype of PPAR receptors, respectively. WO 2010/013071 A2 for example describes sulfonamides which act as PPAR alpha/gamma antagonists. Disadvantageous is however that these compounds are no PPAR beta/delta inhibitors, i.e. do not act as antagonists or as inverse PPAR beta/delta agonists. These effects are of relevance for the treatment of inflammatory diseases, tumor diseases and leukemias.

Aim of the present invention is to overcome the limitations of the prior art by providing new compounds.

This aim is solved according to the present invention by the technical teaching of the independent claims. Further advantageous embodiments of the invention derive from the dependent claims, the descriptions, the figures as well as the examples.

Surprisingly it was found that the sulfonamides of the general formula (I) represent better inhibitors of PPAR beta/delta for the treatment of beta/delta-mediated diseases than compound GSK0660 which is known from the literature to exhibit an inhibitory effect.

The present invention thus relates to compounds of the general formula (I)

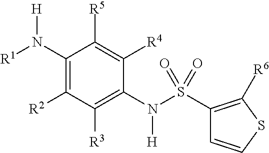

wherein
R¹ represents one of the following groups:
—R⁷, —CH₂—R⁷, —CH(R⁸)—R⁷, —C(R⁹)(R⁸)—R⁷, —CH₂—CH₂—R⁷, —CH(R⁹)—CH(R⁸)—R⁷, —C(R¹¹)(R¹⁰)—C(R⁹)(R⁸)—R⁷, —(CH₂)ₙ—R⁷, —CH₂—R³¹, —CH₂—CH₂—R³¹, —(CH₂)ₙ—R³¹,
R², R³, R⁴, R⁵ independently of one another represent the following groups:
—H, —OH, —OR¹², —OR¹³, —CF₃, —OCF₃, —F, —Cl, —Br, —I, —COR¹⁴, —COR¹⁵, —COOH, —COOR¹⁶, —COOR¹⁷, —CONH₂, —CONH(R¹⁸), —CON(R¹⁹)(R²⁰), —NH₂, —NH(R²¹), —N(R²²)(R²³), —R²⁴, —R²⁵, —OOCR²⁴, —OOCR²⁵, —R²⁶, —R²⁷, —OOC—OR²⁶, —OOC—OR²⁷, —OOC—NH₂, —OOC—NH(R²⁶), —OOC—N(R²⁶)(R²⁷), —NHCO—R²⁸;
R⁶ represents one of the following groups:
—H, —COOH, —CH₂—COOH, —COOR²⁹, —CH₂—COOR²⁹, —OH, —CH₂OH, —OR²⁹, —CH₂OR²⁹, —COR²⁹, —CONH(R²⁹), —CON(R²⁹)(R³⁰), —SH, —SR²⁹, —CH₂—OOCR²⁹, —CH₂—OOC—OR²⁹, —CH₂—OOC—NH₂, —CH₂—OOC—NH(R²⁹), —CH₂—OOC—N(R²⁹)(R³⁰);

R⁷-R³⁰ and R³⁷-R⁴¹ independently of one another represent the following groups:

—CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₂—CH₂-Ph, —CH₂—CH₂—CH₂-Ph, —CH═CH-Ph, —C≡C-Ph, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH═CH—CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, —CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —CH═CH—C(CH₃)₃, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₃H₇)═CH₂, —CH₂—C(C₂H₅)═CH—CH₃, —CH(C₂H₅)—CH═CH—CH₃, —C(C₄H₉)═CH₂, —C(C₃H₇)═CH—CH₃, —C(C₂H₅)═CH—C₂H₅, —C(C₂H₅)═C(CH₃)₂, —C[C(CH₃)₃]═CH₂, —C[CH(CH₃)(C₂H₅)]═CH₂, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH₂—CH═CH—CH═CH—CH₃, —CH═CH—CH₂—CH═CH—CH₃, —CH═CH—CH═CH—C₂H₅, —CH₂—CH═CH—C(CH₃)═CH₂, —CH₂—CH═C(CH₃)—CH═CH₂, —CH₂—C(CH₃)═CH—CH═CH₂, —CH(CH₃)—CH═CH—CH═CH₂, —CH═CH—CH₂—C(CH₃)═CH₂, —CH═CH—CH(CH₃)—CH═CH₂, —CH═C(CH₃)—CH₂—CH═CH₂, —C(CH₃)═CH—CH₂—CH═CH₂, —CH═CH—CH═C(CH₃)₂, —CH═CH—C(CH₃)═CH—CH₃, —CH═C(CH₃)—CH═CH—CH₃, —C(CH₃)═CH—CH═CH—CH₃, —CH═C(CH₃)—C(CH₃)═CH₂, —C(CH₃)═CH—C(CH₃)═CH₂, —C(CH₃)═C(CH₃)—CH═CH₂, —CH—CH—CH═CH—CH═CH₂, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —CH₂—CH(CH₃)—C≡CH, —CH(CH₃)—CH₂—C≡CH, —CH(CH₃)—C≡C—CH₃, —C₄H₈—C≡CH, —C₃H₆—C≡CH₃, —C₂H₄—C≡C—C₂H₅, —CH₂—C≡C—C₃H₇, —C≡C—C₄H₉, —C₂H₄—CH(CH₃)—C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —C≡C—CH₂—CH(CH₃)₂, —C≡C—C(CH₃)₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —C(CH₃)(C₂H₅)—C≡CH, C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —CH₂—C≡C—C≡C—CH₃, —C≡C—CH₂—C≡C—CH₃, —C≡C—C≡C—C₂H₅, —C≡C—CH(CH₃)—C≡CH, —CH(CH₃)—C≡C—C≡CH, —CH(C≡CH)—CH₂—C≡CH, —C(C≡CH)₂—CH₃, —CH₂—CH(C≡CH)₂, —CH(C≡CH)—C≡C—CH₃;
R³¹ represents one of the following groups:

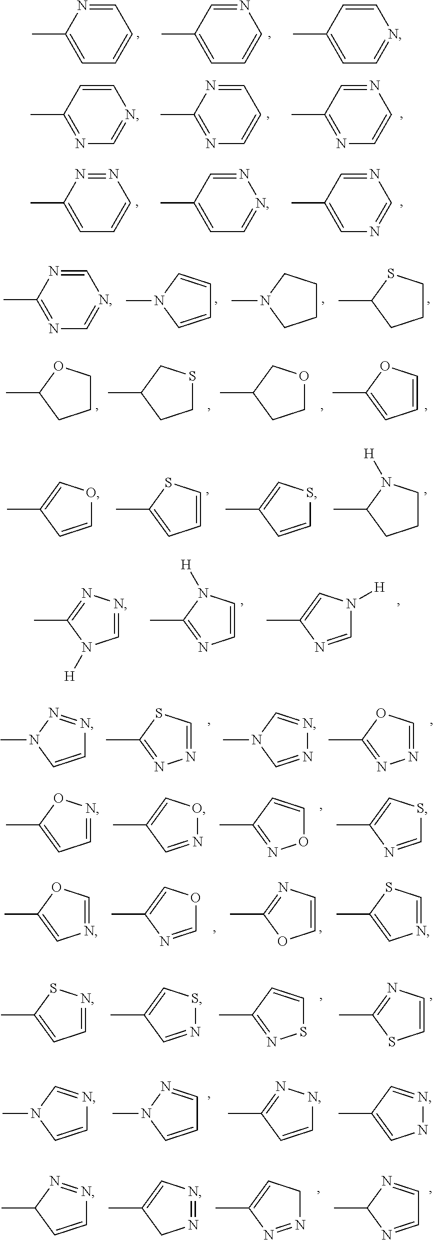

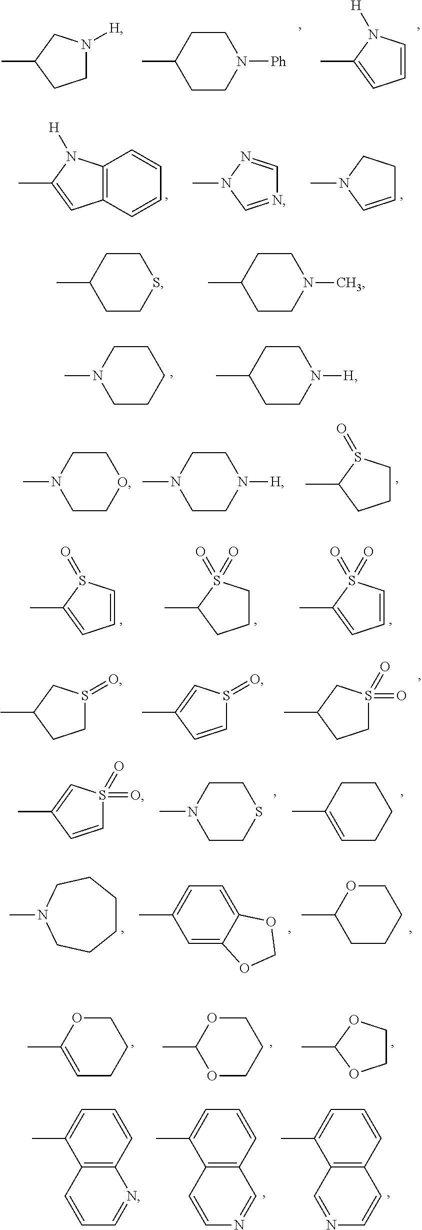

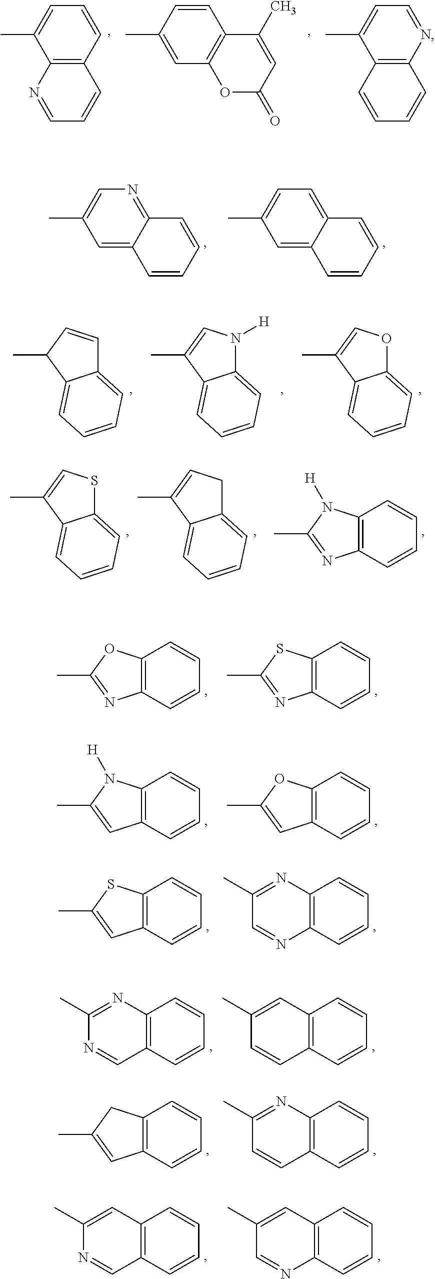

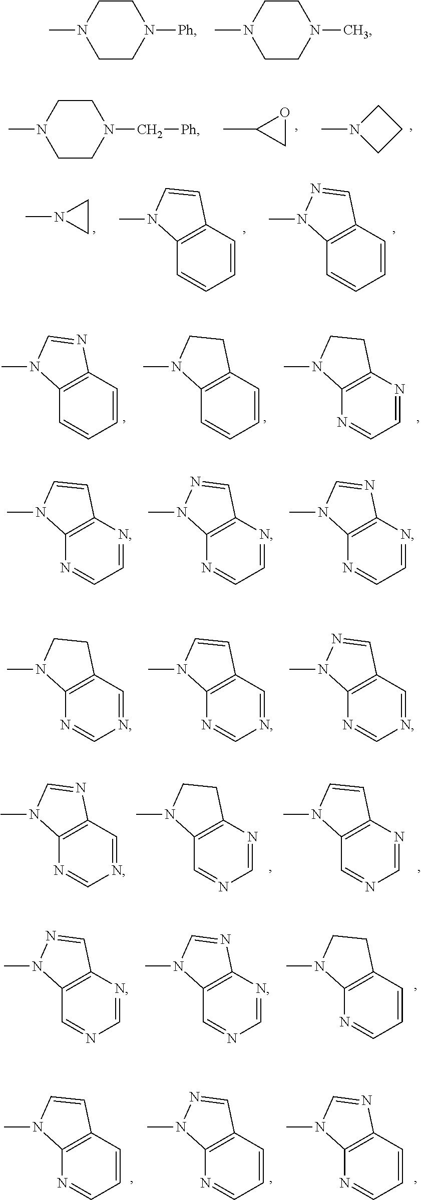

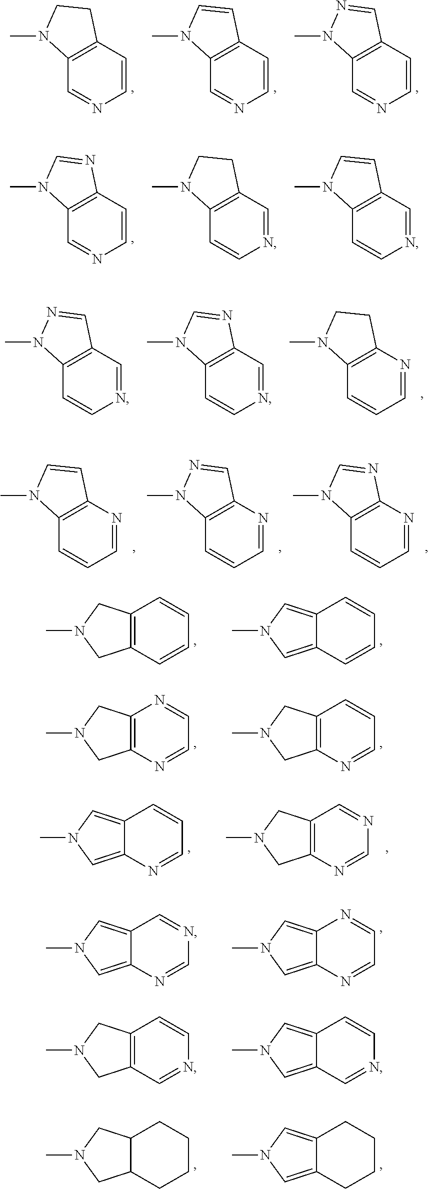

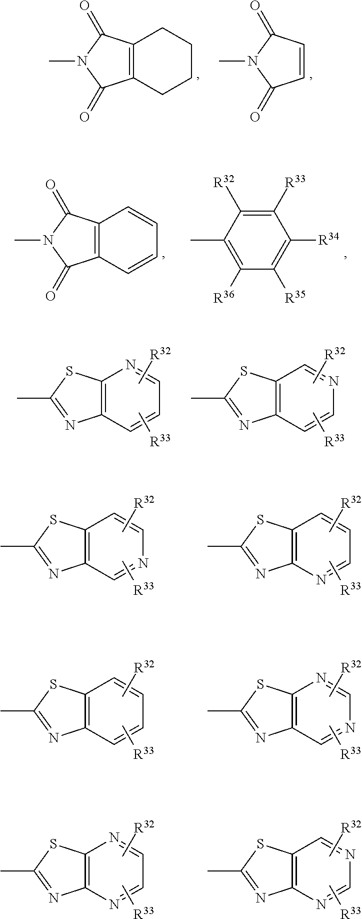

R³²-R³⁶ independently of one another represent the following groups:
—R³⁷, —R³⁸, —R³⁹, —R⁴⁰, —R⁴¹, —H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —P(O)(OH)₂, —P(O)(OCH₃)₂, —P(O)(OC₂H₅)₂, —P(O)(OCH(CH₃)₂)₂, —C(OH)[P(O)(OH)₂]₂, —Si(CH₃)₂(C(CH₃)₃), —Si(C₂H₅)₃, —Si(CH₃)₃, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CONH[CH(CH₃)₂]₂, —CONH[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂N H₂, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —NH—C(═NH)—NH₂, —NH—C(═NH)—NHCH₃, —NH—C(═NH)—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —O—CO—NH₂, —NH—C(═NH)—N[CH(CH₃)₂]₂, —NH—C(═NH)—N[C(CH₃)₃]₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃;
n is an integer, selected from 1, 2, 3, 4 or 5;
as well as metal complexes thereof, salts, enantiomers, enantiomeric mixtures, diastereomers, diastereomeric mixtures, tautomers, hydrates, solvates and racemates of the abovementioned compounds.

Preferred are compounds of the general formula (I), wherein R¹ represents —CH₂—R⁷, —CH(R⁸)—R⁷, —CH₂—CH₂—R⁷, —CH(R⁹)—CH(R⁸)—R⁷, —(CH₂)ₙ—R⁷, —CH₂—R³¹, —CH₂—CH₂—R³¹ or —(CH₂)ₙ—R³¹ and wherein groups R⁷, R⁸, R⁹ and R³¹ have the meaning given above.

Particularly preferred are compounds of the general formula (I), wherein R¹ represents —CH₂—R⁷, —CH₂—CH₂—R⁷, —(CH₂)ₙ—R⁷, —CH₂—R³¹, —CH₂—CH₂—R³¹ or —(CH₂)ₙ—R³¹ and wherein groups R⁷ and R³¹ have the meaning given above.

Preferred are furthermore the following compounds:
3-(4-hexylamino-2-methoxyphenylsulfamoyl)-thiophene-2-carboxylic methyl ester
3-(4-hexylamino-3-methoxyphenylsulfamoyl)-thiophene-2-carboxylic methyl ester
3-[2-methoxy-4-(3-methylbutylamino)-phenylsulfamoyl)-thiophene-2-carboxylic methyl ester
3-(4-benzylamino-2-methoxyphenylsulfamoyl)-thiophene-2-carboxylic methyl ester
3-(4-tert-butylamino-2-methoxyphenylsulfamoyl)-thiophene-2-carboxylic methyl ester
3-[2-methoxy-4-(propylamino)phenylsulfamoyl]thiophene-2-carboxylic methyl ester
3-(4-butylamino-2-methoxyphenylsulfamoyl)-thiophene-2-carboxylic methyl ester
3-[2-methoxy-4-(pentylamino)phenylsulfamoyl]thiophene-2-carboxylic methyl ester
3-[2-methoxy-4-(iso-propylamino)phenylsulfamoyl]thiophene-2-carboxylic methyl ester ester
3-(4-iso-butylamino-2-methoxyphenylsulfamoyl)-thiophene-2-carboxylic methyl ester
3-(4-iso-hexylamino-2-methoxyphenylsulfamoyl)-thiophene-2-carboxylic methyl ester
3-(4-iso-heptylamino-2-methoxyphenylsulfamoyl)-thiophene-2-carboxylic methyl ester
3-(4-cyclohexylamino-2-methoxyphenylsulfamoyl)-thiophene-2-carboxylic methyl ester
3-[2-ethoxy-4-(hexylamino)phenylsulfamoyl]thiophene-2-carboxylic methyl ester
3-(4-decylamino-3-methoxyphenylsulfamoyl)-thiophene-2-carboxylic methyl ester
3-(4-hexylaminophenylsulfamoyl)-thiophene-2-carboxylic methyl ester
3-(4-hexylamino-2-propoxyphenylsulfamoyl)-thiophene-2-carboxylic methyl ester
3-[2-butoxy-4-(hexylamino)phenylsulfamoyl]thiophene-2-carboxylic methyl ester
3-(4-hexylamino-2-pentoxyphenylsulfamoyl)-thiophene-2-carboxylic methyl ester
3-(4-hexylamino-2-iso-propoxyphenylsulfamoyl)-thiophene-2-carboxylic methyl ester
3-(4-hexylamino-2-iso-pentoxyphenylsulfamoyl)-thiophene-2-carboxylic methyl ester
3-[4-hexylamino-2-(2-phenylethoxy)phenylsulfamoyl]-thiophene-2-carboxylic methyl ester

Surprisingly it was found that the amino group substituted with R¹ should advantageously be a secondary and no tertiary amino group, i.e. should carry a proton instead of a third substituent.

Furthermore became evident that R¹ should advantageously carry a methylene group in the alpha position and that the alpha carbon atom should advantageously be a secondary instead of tertiary or quaternary carbon atom. In addition it was found that the alpha carbon atom in R¹ should not be part of an aromatic or unsaturated ring system as in the case of the well-known compound GSK0660.

Compounds of the present invention of the general formula (I) can be obtained according to the following synthesis.

As starting materials serve commercially available nitrobenzenes as well as nitrobenzenes producible according to common syntheses with a leaving group in the para position or para-nitroanilines as well as appropriately 2-substituted thiophene-3-sulfonylchlorides. The synthesis of compounds of this invention of the general formula (I) follows the synthesis description according to steps A) [A1) or A2)], B) and C).
A1) Synthesis of a secondary para-nitroaniline from the corresponding primary para-nitroaniline by conversion with a nucleophile of the general formula R¹-LG*, wherein LG* represents a leaving group, with elimination of LG*-H which optionally can be scavenged using a base under formation of the corresponding salt.

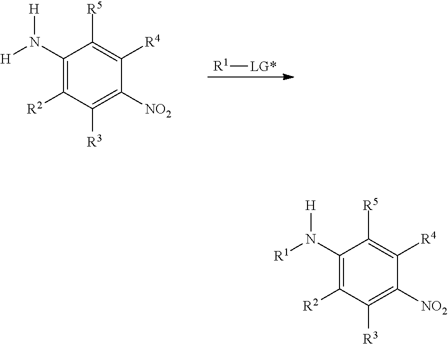

or
A2) Synthesis of a secondary para-nitroaniline from the corresponding nitrobenzene with a leaving group LG in the para position relative to the nitro group by conversion with a primary amine of the general formula R¹—NH₂, with elimination of LG-H which optionally can be scavenged using a base under formation of the corresponding salt.

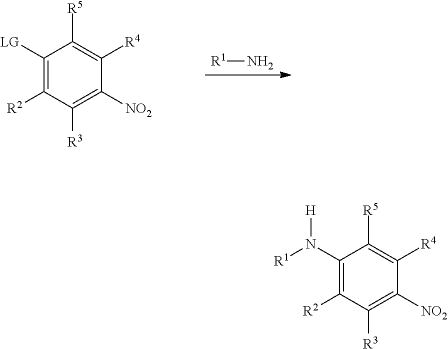

and
B) Reduction of the nitro group of the secondary para-nitroaniline obtained according to step A1) or A2) to the amino group

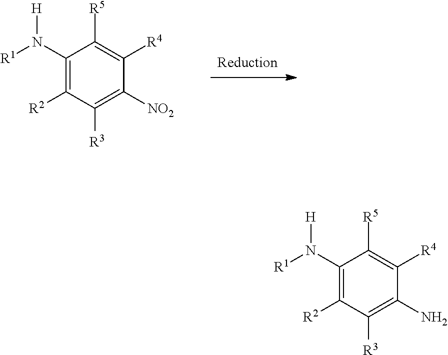

and
C) Conversion of the para-aminoaniline obtained according to step B) using a thiophene-3-sulfonylchloride under alkaline conditions into compounds of the general formula (I).

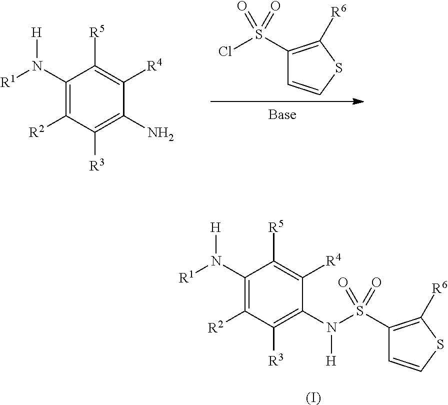

Step A1) can be carried out in a solvent mixture composed of tert-butanol and DMSO using KOH as base and an alkyl halide as nucleophile, preferably at elevated temperature or under reflux.

In step A2), the leaving group LG is advantageously fluorine. As base, preferably carbonates like K₂CO₃, alkanolate like e.g. tert-butanolate (t-BuO⁻) or tertiary amines like for example triethylamine are utilized. The substitution reaction may furthermore be carried out at elevated temperature and/or under pressure and/or utilizing microwaves. A preferred solvent is acetonitrile.

Comparative compound 2 is synthesized according to synthesis steps A2), B) and C), whereby R¹—NH₂ in this case represents morpholine.

The reduction to the amine according to step B) is preferably carried out using hydrogen gas and Pd/C as catalyst. It may be advantageous to perform the reaction in the absence of visible light. The reaction mixture is preferably stirred under hydrogen atmosphere for 12 h to 48 h at room temperature.

Step c) is preferably carried out in pyridine with addition of dimethylaminopyridine at room temperature for 12 h to 48 h or in methylene chloride under addition of triethylamine as base at 0° C., followed by 10 h to 20 h at room temperature. Exclusion of light may be advantageous.

The experimental efficacy of the compounds is described in the following. Substances of this invention bind in vitro to the ligand binding domain of the PPAR beta/delta receptor. Said substances compound 1, compound 2, compound 3, compound 4 and compound 5 exhibit significant competition efficiency in the TR-FRET ligand binding assay as compared to the fluorescent ligand Fluormone® Pan-PPAR-Green. The transcriptional activity of PPAR beta/delta in human WPMY-1 myofibroblasts after activation by agonist L165041 is inhibited in a PPAR subtype-specific manner by the substances of this invention compound 1, compound 2, compound 3 and compound 5 (example 24).

The substances of this invention compound 1, compound 2, compound 3 and compound 4 furthermore induce in vitro the interaction of the ligand binding domain of the PPAR beta/delta receptor with a synthetic peptide fragment of the commonly known corepressor SMRT (SMRT-ID2). Expression of the ANGPTL4 gene coding for the angiopoietin-like protein 4 (ANGPTL4) is induced by PPAR beta/delta or other stimuli. ANGPTL4 is assumed to be involved in tumor growth, tumor progression and tumor metastasis. Using said substances of this invention compound 1, compound 2, or compound 3, the basal expression of ANGPTL4 in human primary fibroblasts WI-38, WPMY-1 myofibroblasts and peritoneal macrophages is reduced significantly by at least 50%. Mean inhibitory concentration (IC₅₀) values of the substances of this invention are in a range between 1 and 30 nM. The induction of ANGPTL4-expression by stimuli known to the expert tumor growth factor-beta (TGF-beta1, TGF-beta2), dexamethasone or TPA [12-O-tetradecanoylphorbol-13-acetate)=phorbol-12-myristate-13-acetate (PMA)] in human fibroblasts can furthermore significantly be reduced by the substance compound 1 of this invention. If cells of the breast cancer cell line MDA-MB-231-luc21H4 are treated with compound 1, this also leads to a drastic reduction of ANGPTL4 expression. These features classify the substances compound 1, compound 2, compound 3 and compound 4 of this invention as inhibitor and in particular as inverse agonists of the PPAR beta/delta receptor.

The substance compound 5 of this invention prevents both the recruitment of the co-activator peptide C33 to the ligand binding domain of the PPAR beta/delta receptor which is induced by a well-known synthetic agonist, as well as the interaction of the ligand binding domain of the PPAR beta/delta receptor with corepressor peptide SMRT-ID2 which is mediated by said substance compound 1 of this invention. These features classify said substance compound 5 of this invention as inhibitor and in particular as competitive antagonist of the PPAR beta/delta receptor.

The present invention furthermore also concerns compounds of the general formula (I) to be used as inhibitors or antagonists, particularly preferred as competitive antagonists or inverse agonists of a receptor of the PPAR beta/delta type.

Within the sense of the present invention, an agonist is understood to be a compound which activates signal transduction and/or transcription in the respective cell by occupation of a receptor.

Antagonists are understood to be compounds which are, due to preferential interaction with the inactive receptor, able to prevent or inhibit the activation of this receptor by an agonist. A competitive antagonist competes with an agonist for binding to a receptor and can be displaced by higher agonist concentrations according to the law of mass action. A competitive antagonist results in a parallel shift of the dose-effect curve of an agonist. Within the context of the present invention, these are advantageously compounds which prevent and/or inhibit the binding of an agonist and thus of a co-activator without binding of a corepressor.

Inverse agonists herein are compounds which bind to a receptor with constitutive activity and reduce the activity thereof. An inverse agonist leads—in contrast to a full agonist, thus to a negative effect or a pharmacological effect which is opposite to the effect exhibited by an agonist. Within the context of the present invention, these are advantageously compounds which cause or promote the binding of a corepressor.

PPAR beta/delta inhibitors are relevant for a use in medicine, since PPAR beta/delta has increasingly been linked in the past years with severe diseases like atherosclerosis, diabetes type II, lipid metabolism disorders as well as several tumor diseases and leukemias.

The present invention therefore also concerns the use of compounds of the general formula (I) in medicine.

Compounds of the present invention are suited for the treatment and/or prevention of diseases in which inflammatory processes, inflammations or cell differentiation processes are involved, and for the treatment of proliferative diseases. Such diseases are for example, but not limited to, atherosclerosis like for example coronary sclerosis including angina pectoris or myocardial infarction, vascular restenosis or reocclusion, chronic inflammatory bowel diseases like for example Crohn's disease and ulcerative colitis, pancreatitis, other inflammatory states like retinopathy. Further inflammatory diseases which are influenced by PPAR beta/delta are for example polycystic ovary syndrome, asthma, osteoarthritis, lupus erythematosus (LE) or inflammatory rheumatic diseases like for example rheumatoid arthritis, vasculitis, cachexia, gout, ischemia, reperfusion syndrome and respiratory distress syndrome. Erythemato-squamous dermatoses like for example psoriasis and acne vulgaris. Further skin diseases which are influenced by PPAR-beta/delta and for this reason count among the possible diseases which can be treated using compounds of the formula (I) are: eczemas and neurodermatitis, dermatites like for example seborrheic dermatitis or photodermatitis, keratitis and keratoses like for example seborrheic keratoses, senile keratosis, actinic keratosis, light-induced keratoses, keratosis pilaris proliferations, warts including condylomata or condylomata acuminata, human papillomavirus infections (HPV) like for example genital tract papillomas, viral warts like for example molluscum contagiosum, leukoplakia papular dermatoses like for example lichens, skin cancer like for example basal cell carcinomas, melanomas or cutaneous T-cell lymphomas, local benign epidermal tumors like for example keratoderma, epidermal nevus and lumps, panniculitis, conjunctivitis, balanitis, intertrigo, vaginitis, cheilitis, sun burn, alopecia areata.

The proliferative diseases concerned are primarily tumors such as e.g. benign tumors, cancer and metastases as well as inflammation-related diseases like for example arthritis or psoriasis. The tumor diseases can be chosen from the group including or consisting of: sarcomas (like e.g. liposarcomas), carcinomas (like e.g. of the gastrointestinal tract, the liver, the bile duct and the pancreas, the lungs, the urogenital tract, the mammary glands etc.), endocrine tumors, acute and chronic leukemias and other myeloproliferative diseases and lymphomas.

Compounds of the present invention of the formula (I) are also suitable for the treatment of neurodegenerative diseases like e.g. Alzheimer's disease and Parkinson's disease.

Said compounds of the present invention of the formula (I) are also suitable for the treatment of liver diseases like steatosis, steatohepatitis and hepatitis. Said compounds of the present invention of the formula (I) are furthermore suitable for the treatment of diseases of the fatty acid metabolism and the glucose metabolism in which insulin resistance is involved. Among these count hyperlipidemia, hypertriglyceridemia, hypercholesterolemia. To these diseases furthermore belongs diabetes mellitus, in particular type 2 diabetes including the prevention of associated sequelae like for example hyperglycemia, increase of insulin resistance, glucose homeostasis disorders, protection of pancreatic beta-cells, prevention of macro- and microvascular diseases. Furthermore belong to this group dyslipidemias and consequences thereof like for example atherosclerosis, coronary heart diseases, cerebrovascular diseases, in particular those which are characterized by the following factors: high plasma triglyceride concentrations, high postprandial plasma triglyceride concentrations, low HDL cholesterol concentrations, low ApoA lipoprotein concentrations, high LDL cholesterol concentrations, LDL cholesterol particles with low density, high ApoB lipoprotein concentrations. Various other diseases may be associated with the metabolic syndrome: obesity, thromboses, hypercoagulable and prothrombotic states (arterial and venous), high blood pressure, heart failure like for example, but not limited to, myocardial infarction, hypertensive heart disease or cardiomyopathy.

The present invention furthermore concerns pharmaceutical compositions which were prepared utilizing at least one compound of this invention or a salt thereof. In addition to at least one compound of the general formula (I), said pharmaceutical compositions contain a pharmacologically acceptable carrier, excipient and/or solvent.

Depending on the substituents (e.g. carboxyl groups) on the sulfonamide compounds of this invention, these also form pharmaceutically acceptable salts with organic and inorganic bases. Examples for suitable bases for such salt formation are for example NaOH, KOH, NH₄OH, tetraalkylammonium hydroxide and the like which are known to the expert in this field.

The sulfonamide compounds of the present invention are alkaline and can form salts with acids. Suitable acids for acid addition salt formation are for example hydrochloric acid, hydrobromic acid, sulphuric acid, phosphoric acid, acetic acid, citric acid, oxalic acid, malic acid, salicylic acid, p-amino salicylic acid, malonic acid, fumaric acid, succinic acid, ascorbinic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethane sulfonic acid, ethylenesulfonic acid, p-toluenesulfonic acid, naphthyl sulfonic acid, sulfanilic acid, camphorsulfonic acid, quinic acid, mandelic acid, o-methyl mandelic acid, hydrogen benzene sulfonic acid, picrinic acid, adipic acid, d-o-tolyl tartaric acid, tartronic acid, α-toluylic acid, (o-, m-, p-) toluylic acid, naphthylamine sulfonic acid and other mineral or carboxylic acids known to the expert in this field. It is furthermore possible to form acid addition salts of the sulfonamide compounds of this invention with amino acids like methionine, tryptophan, lysine or arginine.

Compounds of the general formula (I) can furthermore be administered in the form of their pharmaceutically acceptable salts and, if required, in combination with in general non-toxic pharmaceutically acceptable carriers, excipients or diluents. Medications of the present invention are prepared in a conventional solid or liquid carrier or in diluents and a conventional excipient manufactured by pharmaceutically means and with appropriate dosage in a known manner. Preferred are preparations suitable for oral delivery. Dosage forms to be delivered orally include for example pills, tablets, layer tablets, film tablets, coated tablets, capsules, powders and deposits.

Preferred dosage forms of delivery are tablets, film tablets, coated tablets, gelatine capsules and opaque capsules. Each pharmaceutical composition contains at least one compound of the general formula (I) and advantageously at least one of the compounds 1-4 and/or pharmaceutically acceptable salts thereof in an amount of 50 mg to 150 mg, preferred 80 mg to 120 mg and most preferred in an amount of 100 mg per formulation.

The subject-matter of the present invention furthermore includes pharmaceutical preparations for parenteral including dermal, intradermal, intragastral, intracutaneous, intravasal, intravenous, intramuscular, intraperitoneal, intranasal, intravaginal, intrabuccal, percutaneous, rectal, subcutaneous, sublingual, topic or transdermal application, which contain in addition to typical vehicles and diluents a sulfonamide compound of the general formula (I) and/or a pharmaceutically acceptable salt thereof as active ingredient.

Within the context of the disclosed procedures, pharmaceutical compositions of this invention containing compounds of the general formula (I) as active ingredients are typically administered in a mixture with suitable carrier materials, chosen with respect to the intended form of delivery, i.e. oral tablets, capsules (filled either solid, semi-solid or liquid-filled), powders for compositions, oral gels, elixirs, dispersible granulates, syrups, suspensions and the like and consistent with conventional pharmaceutical practices. For oral delivery in the form of tablets or capsules for example, the active compound may be combined with any oral non-toxic pharmaceutically acceptable inert carrier like lactose, starch, sucrose, cellulose, magnesium stearate, dicalcium phosphate, calcium sulfate, talcum, mannitol, ethyl alcohol (liquid forms) and the like. When requested or required, suitable binding agents, lubricants, disintegrants and coloring agents may be also added to the mixture. Powders and tablets may consist of approximately 5 to up to 95 percent of the composition of the present invention.

Suitable binding agents include starch, gelatin, natural sugars, corn sweetener, natural and synthetic gums like acacia gum, sodium alginate carboxymethyl cellulose, polyethylene glycol and waxes. Among the lubricants utilized for these dosage delivery forms are to be mentioned boric acid, sodium benzoate, sodium acetate, sodium chloride and the like. Disintegrants include starch, methyl cellulose, guar gum and the like. Sweeteners, flavors and preservatives may, if beneficial, also be included. Some of the above mentioned expressions, namely disintegrants, diluents, lubricants, binding agents and the like are discussed in more detail in the following.

Said compositions according to the present invention may additionally be formulated in a delayed release dosage form to allow a time-controlled release of one or more compounds of the general formula (I) and to optimize the therapeutic efficacy thereof. Suitable dosage forms for delayed release include layer tablets composed of layers with different erosion velocities or polymer matrices with controlled release features which are impregnated with the active compound and designed in the form of tablets or capsules which contain such impregnated or encapsulated porous polymer matrices.

Liquid form preparations include solutions, suspensions and emulsions. Exemplarily mentioned here are water or water-propylene glycol solutions for parenteral injections or the addition of sweeteners and opacifiers for oral solutions, suspensions and emulsions. Preparations in liquid form may furthermore include solutions for intranasal delivery.

Aerosol preparations suitable for inhalation may include solutions and solids in powder form which may be combined with a pharmaceutically acceptable carrier like for example a compressed inert gas, e.g. nitrogen.

For the preparation of suppositories, initially a low-melting wax like for example a mixture of fatty acid glycerides such as e.g. cocoa butter is melted and the active ingredient is dispersed therein homogeneously by stirring or similar ways of mixing. The molten homogeneous mixture is then poured into adequately dimensioned molds, allowed to cool and thus solidified.

Also included are preparations in solid form which are to be converted immediately prior to use into preparations in liquid form for either oral or parenteral application. Such liquid forms include solutions, suspensions and emulsions.

The sulfonamide compounds of the present invention can furthermore be transdermally applicable. Transdermal compositions can take the form of creams, lotions, aerosols and/or emulsions and can be incorporated in a transdermal patch of the matrix or reservoir type commonly used by the state of the art for this purpose.

The term capsule refers to a particular container or enclosure manufactured from methyl cellulose, polyvinyl alcohols or denatured gelatins or starch for the purpose of holding or including compounds which comprise active ingredients. Coated hard capsules are typically manufactured from a mixture of bone and pig skin gelatins with relatively high gel strength. The capsule itself may contain small amounts of coloring agents, opacifiers, plasticizers and preservatives.

Tablet refers to compressed or poured solid dosage forms which contains the active ingredients together with suitable diluents. Tablets may be manufactured by compression of mixtures or granulates obtained by means of wet granulation, dry granulation or by compacting known to the expert in this field.

Oral gel refers to active ingredients which are dispersed or solubilized in a hydrophilic semi-solid matrix.

Powder used for compositions refers to powder mixtures containing the active ingredients and suitable diluents to be suspended in water or saps.

Suitable diluents are substances which generally account for the major part of the composition or dosage form. Suitable diluents include sugars such as lactose, sucrose, mannitol and sorbitol, starches derived from wheat, maize, rice and potatoes and celluloses like micro-crystalline cellulose. The amount of diluent in the composition may vary and range from approximately 5 to approximately 95% by weight of the total composition, preferred from approximately 25 to approximately 75% by weight, and even more preferred from approximately 30 to approximately 60% by weight.

The term disintegrant refers to materials which are added to the composition in order to promote the break up (dissolution) and release of the medicaments. Suitable disintegrants include starches, cold water-soluble modified starches like sodium carboxymethyl starch, natural and synthetic gums like locust bean gum flour, karaya, guar, tragacanth and agar, cellulose derivatives like methyl cellulose and sodium carboxymethyl cellulose, micro-crystalline celluloses and cross-linked micro-crystalline celluloses like sodium croscarmellose, alginates like alginic acid and sodium alginate, alumina like betonite and foaming mixtures. The amount of disintegrant in the composition may vary and range from approximately 2 to approximately 20% per weight of the composition and more preferred from approximately 5 to approximately 10% per weight.

Binding agents characterize substances which give adhesiveness to or “glue together” powder particles or render these cohesive by forming granulates and thus serve as binders in said formulations. Binding agents add to the cohesive strength which is already available in the diluents or the raising agent. Suitable binding agents include sugars like sucrose, starches derived from wheat, maize, rice and potatoes, natural gums like acacia gum, gelatin and tragacanth, seaweed-derivates like alginic acid, sodium alginate and ammonium calcium alginate, cellulose materials like methyl cellulose and sodium carboxymethyl cellulose and hydroxypropyl methyl cellulose, polyvinylpyrrolidone and inorganic compounds like magnesium aluminum silicate. The amount of binding agent in the composition may vary and range from approximately 2 to approximately 20% per weight of the composition, preferred from approximately 3 to approximately 10% per weight, and even more preferred from approximately 3 to approximately 6% per weight.

Lubricant refers to a substance added to the dosage form to facilitate the release of tablets, granulates etc. after compression from the mold or die by means of reducing the friction. Suitable lubricants include metal stearates like magnesium stearate, calcium stearate or potassium stearate, stearic acid, high melting point waxes, water-soluble lubricants like sodium chloride, sodium benzoate, sodium acetate, sodium oleate, polyethylene glycol and D,L-leucine. Lubricants are generally added just prior to the compression step since these have to be deposited on the granulate surface between granulate and the parts of the tablet press. The amount of lubricant in the composition may vary and range from approximately 0.2 to approximately 5% per weight of the composition, preferred from approximately 0.5 to approximately 2% per weight, and even more preferred from approximately 0.3 to approximately 1.5% per weight.

Glidants are excipients to prevent adherence (caking) of the material and to improve the flow characteristics of granulates in order to make the flow smooth and uniform. Suitable glidants include silicon dioxide and talcum. The amount of glidant in the composition may vary and range from approximately 0.1 to approximately 5% per weight of the composition, preferred from approximately 0.5 to approximately 2% per weight.

Coloring agents are excipients to add a coloring to the composition or the dosage form. Such excipients may include food grade dyes which are adsorbed to a suitable adsorbent like for example alumina or aluminum oxide. The amount of coloring agent in the composition may vary and range from approximately 0.1 to approximately 5% per weight of the composition, preferred from approximately 0.1 to approximately 1% per weight.

As used herein, the term “pharmaceutically effective amount” of an inhibitor refers to an amount which is sufficiently high to obtain the desired physiological result either in in vitro treated cells or in a patient treated in vivo. A pharmaceutically effective amount specifically refers to an amount which is sufficiently high to inhibit and/or activate for a certain period of time one or more of the clinically defined pathologic processes which are associated with a PPAR beta/delta receptor. The pharmaceutically effective amount may vary depending on the specific inhibitor used and furthermore depends on a variety of factors and conditions related to the subject to be treated and the respective severity of the disease. If for example an inhibitor is to be administered in vivo, factors like age, weight and health status of the patient as well as dose response curves and toxicological data obtained from pre-clinic studies count among the important factors to be considered. In the case that the inhibitor is intended to be brought into contact with cells in vivo, a variety of pre-clinic in vitro studies is to be designed to assess parameters such as uptake kinetics, biological half-life, dosage, toxicity and the like. The determination of a pharmaceutically effective amount for a given pharmaceutically active substance is entirely within the ability of the expert in this field.

It is easily apparent to the expert that other suitable modifications and adaptations of herein described compositions are obvious and can be carried out without departing from the herein disclosed scope of protection of the invention or the embodiments.

The present invention furthermore concerns pharmaceutical compositions comprising at least one compound of the general formula (I) for the treatment of inflammatory processes, inflammations, cell differentiation processes, proliferative diseases, tumors, metastases, cancer, liver diseases as well as disorders of the fatty acid metabolism and the glucose metabolism in which insulin resistance is involved.

Preferred embodiments of the present invention are described in the following, whereby the invention includes all preferred embodiments listed below alone or on combination with one another.

### FIGURE LEGENDS

FIG. 1: Competitive in vitro ligand binding to PPAR beta/delta.
The displacement of ligand Fluormone® Pan-PPAR Green from recombinant GST-PPAR beta/delta ligand binding domain (LBD) by the substances of the present invention comparative compound (CC) 2, compound 1 (A), compound 2 (B), and compound 3 (C), compound 4 (D), compound 5 (E) is determined by TR-FRET analysis. Shown is the ratio of fluorescence intensity at 520 nm (fluorescein emission, excited by terbium emission) and 495 nm (terbium emission). All data points represent average values of triplicates (+/−standard deviation).

FIG. 2: Induction of corepressor peptide binding to GST-PPAR beta/delta ligand binding domain (LBD) in vitro in dependency of the concentration of the substances of this invention comparative compound (CC) 2, compound 1 (A), compound 2 (B), compound 3 (C), compound 4 (D) and compound 5 (E). The interaction of fluorescein-labeled SMRT-ID2 peptide with recombinant GST-PPAR beta/delta LBD bound to a terbium-labeled anti-GST antibody is determined by TR-FRET analysis. Shown is the ratio of fluorescence intensity at 520 nm (fluorescein emission, excited by terbium emission) and 495 nm (terbium emission). All data points represent average values of triplicates (+/−standard deviation).

FIG. 3: Inhibition of agonist-induced co-activator peptide binding (A) and inverse agonist compound 1-induced corepressor peptide binding (B) by the substance of this invention compound 5.
The recruitment of the co-activator or corepressor peptide, respectively, is determined in a TR-FRET assay. As co-activator peptide, C33 is utilized, as corepressor peptide SMRT-ID2. The experiment is performed with a constant concentration of 1 μM of the substance of this invention compound 5 and increasing concentrations of the agonist L165041 (A) or the inverse agonist compound 1 (B), respectively.

FIG. 4: Effect of the substances of this invention comparative compound (CC) 2, compound 1, compound 5, compound 2 and compound 3 on the agonist-induced transcriptional activity of PPAR subtypes alpha (A), beta/delta (B) and gamma (C).
WPMY-1 cells are transiently transfected with a luciferase reporter plasmid. Four hours after transfection, cells are treated for 48 hours with the respective antagonist (500 nM), followed by a treatment with 300 nM of PPAR alpha agonist GW7647 (A), 500 nM of PPAR beta/delta agonist L165041 (B) or 300 nM of PPAR gamma agonist GW1929 (C). The relative induction is represented by the luciferase activity of the agonist-treated transfected cells in relation to solvent-treated transfected cells. Asterisks (**, *) indicate significant differences to untreated cells (after t-test; **: p<0.01; *: p<0.05). GW7647 is commercially available from Sigma-Aldrich (Steinheim, Germany). L165041 and GW1929 can be obtained from Biozol (Eching, Germany).

FIG. 5: Influence of the substances of this invention comparative compound (CC) 2, compound 1, compound 5, compound 2 and compound 3 on ANGPTL4 gene expression.
(A) Comparison of relative ANGPTL4 expression, (B) mean inhibitory concentration of substances of this invention. (A) Human myofibroblasts (WPMY-1) are treated for 24 hours with the substances of this invention (1 μM), then RNA is isolated, the cDNA synthesized and analyzed by qPCR, normalized to L27 gene expression. (B) Titration of the effect of substances of this invention on the endogenous ANGPTL4 expression in WPMY-1 cells (left) or peritoneal mouse macrophages (right) after cells have been treated for six hours with the substances of this invention compound 1 and compound 2, respectively; RNA isolation, cDNA synthesis and qPCR as described for (A). The relative expression is calculated in relation to DMSO-treated cells. All values are average values of triplicates (+1-standard deviation). Asterisks (***, **, *) indicate significant differences to DMSO-treated cells (***: p<0.001 after t-test; *: p <0.05).

FIG. 6: Influence of the substances of this invention compound 1 and compound 5 on the induction of ANGPTL4 expression by TGF-beta2 in the breast cancer cell line MDA-MB-231.
The human breast cancer cell line MDA-MB-231 is pre-treated for 24 hours with 1 μM of the substances of this invention compound 1 or compound 5, respectively, and subsequently stimulated with TGF-beta2 (2 ng/ml) for 6 hours. The relative ANGPTL4 expression is determined by qPCR.

FIG. 7: Shows two comparative compounds and 5 selected compounds of this invention according to the general formula (I).

### EXAMPLES

### Example 1

### Comparative Compound 2

### methyl 3-(N-(2-methoxy-4-morpholinophenyl)sulfamoyl)thiophene-2-carboxylate

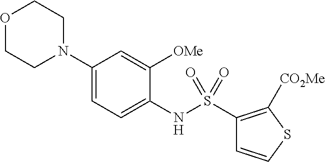 [SMILES: COC(=O)c1sccc1S(=O)(=O)Nc1ccc(N2CCOCC2)cc1OC]

### Step 1

### 4-(3-methoxy-4-nitrophenyl)morpholine

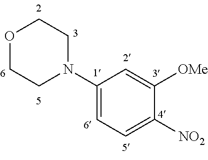 [SMILES: COc1cc(N2CCOCC2)ccc1[N+](=O)[O-]]

4-Fluoro-2-methoxy-1-nitrobenzene (171 mg, 1.00 mmol), morpholine (174 mg, 2.00 mmol, 2.0 eq.) and triethylamine (0.56 ml, 405 mg, 4.00 mmol, 4.0 eq.) were dissolved in acetonitrile (3 ml) and heated for three hours in a sealed reaction vial in a microwave reactor (P≦300 watt) to 120° C. The solvent was subsequently removed under reduced pressure and the residual was dissolved in aqueous dilute hydrochloric acid (5%) and ethyl acetate. The aqueous phase was extracted with ethyl acetate, the combined organic phases were washed with saturated aqueous NaCl solution, dried over Na₂SO₄, filtered, and the solvent was removed under reduced pressure. The raw product was adsorbed to silica gel and purified by column chromatography (isohexane:ethyl acetate=gradient from 2:1 to 1:1) to yield the title compound (188 mg, 79%, yellow solid matter). ¹H-NMR (399.788 MHz, CDCl₃): δ=8.01 (d, ³J_{H,H}=9.4 Hz, 1H, 5′-H), 6.43 (dd, ³J_{H,H}=9.4, ⁴J_{H,H}=2.5 Hz, 1H, 6′-H), 6.35 (d, ³J_{H,H}=2.5 Hz, 1H, 2′-H), 3.96 (br. s, 3H, OCH₃), 3.87 (br. t, ³J_{H,H}=4.9 Hz, 4H, 2-H₂, 6-H₂), 3.35 (br. t, ³J_{H,H}=4.9 Hz, 4H, 3-H₂, 5-H₂). MS (ESI): m/z (%)=239 (100) [M+H]⁺, 261 (41) [M+Na]⁺. HRMS (EI): calculated: 238.095357. found: 238.093980 [M]⁺.

### Step 2

### 2-methoxy-4-morpholinoaniline

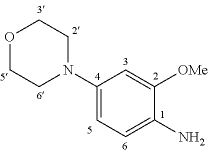 [SMILES: COc1cc(N2CCOCC2)ccc1N]

4-(3-Methoxy-4-nitrophenyl)morpholine (119 mg, 0.500 mmol) was dissolved in ethyl acetate (10 ml), followed by addition of Pd/C (10% w/w, 13 mg). The gaseous phase was subsequently removed several times by evacuation and replaced by hydrogen. The reaction mixture was stirred for 24 h at room temperature in the absence of light, filtered through silica gel and the solvent was removed under reduced pressure. The light-sensitive raw product was directly used for the next synthesis step without further purification.

### Step 3

### methyl 3-(N-(2-methoxy-4-morpholinophenyl)sulfamoyl)thiophene-2-carboxylate

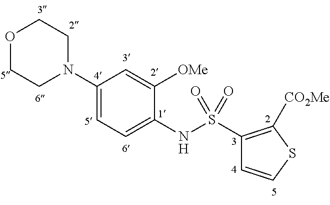 [SMILES: COC(=O)c1sccc1S(=O)(=O)Nc1ccc(N2CCOCC2)cc1OC]

2-Methoxy-4-morpholinoaniline (total amount resulting from the previous synthesis step), dimethylaminopyridine (31 mg, 0.25 mmol, 0.5 eq.) and 3-(chlorosulfonyl)-thiophene-2-carboxylic methyl ester (132 mg, 0.550 mmol, 1.1 eq.) were dissolved together in pyridine (10 ml) and stirred for 48 h at room temperature in the dark. The solvent was removed under reduced pressure and the residual redissolved in dichloromethane and dilute aqueous hydrochloric acid (5%). The aqueous phase was extracted with dichloromethane and the combined organic phases were washed with saturated aqueous NaCl solution, dried over Na₂SO₄, filtrated and the solvent was removed under reduced pressure. The raw product was adsorbed to silica gel and purified using column chromatography (isohexane: ethyl acetate=gradient from 2:1 to 1:1) to yield the title compound (164 mg, 80% over two steps, yellow solid matter). ¹H-NMR (399.788 MHz, CDCl₃): δ=8.26 (br. s, 1H, SO₂NH), 7.41 (d, ³J_{H,H}=8.7 Hz, 1H, 6′-H), 7.39 (d, ³J_{H,H}=5.3 Hz, 1H, 5-H), 7.37 (d, ³J_{H,H}=5.3 Hz, 1H, 4-H), 6.43 (br. d, ³J_{H,H}=8.7 Hz, 1H, 5′-H), 6.26 (br. s, 1H, 3′-H), 4.00 (s, 3H, CO₂CH₃), 3.82 (t, ³J_{H,H}=4.2 Hz, 4H, 3″-H₂, 5″-H₂), 3.56 (s, 3H, OCH₃), 3.09 (t, ³J_{H,H}=4.6 Hz, 4H, 2″-H₂, 6″-H₂). MS (ESI): m/z (%)=208 (18) [M−C₆H₄O₄S₂]⁺, 413 (100) [M+H]⁺, 435 (85) [M+Na]⁺, 826 (31) [2 M+H]⁺, 847 (48) [2 M+Na]⁺. HRMS (EI): calculated: 412.076280. found: 412.074783 [M]⁺. CHN: calculated: 49.50% C, 4.89% H, 6.79% N. found: 49.63% C, 4.90% H, 6.66% N.

### Example 2

### Compound 1

### methyl 3-(N-(4-(hexylamino)-2-methoxyphenyl)sulfamoyl)thiophene-2-carboxylate

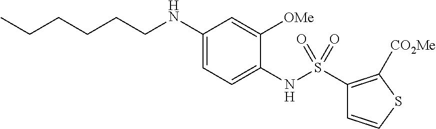 [SMILES: CCCCCCNc1ccc(NS(=O)(=O)c2ccsc2C(=O)OC)c(OC)c1]

### Step 1

### N-hexyl-3-methoxy-4-nitroaniline

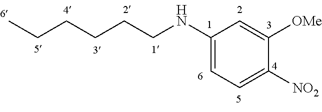 [SMILES: CCCCCCNc1ccc([N+](=O)[O-])c(OC)c1]

4-Fluoro-2-methoxy-1-nitrobenzene (180 mg, 1.05 mmol) was dissolved together with K₂CO₃ (180 mg, 1.30 mmol, 1.2 eq.) in acetonitrile (20 ml), prior to the addition of hexylamine (330 μl, 253 mg, 2.50 mmol). The reaction mixture was heated for 48 h under reflux, followed by cooling to RT and removal of the solvent under reduced pressure. The residual was redissolved in ethyl acetate and subsequently washed with H₂O, aq. HCl solution (0.1 M) and sat. aq. NaCl solution. The organic phase was dried over Na₂SO₄, filtered and the solvent was removed under reduced pressure. Column chromatography purification (isohexane:ethyl acetate=4:1) yielded the title compound (211 mg, 80%) as yellow solid matter. ¹H-NMR (399.788 MHz, CDCl₃): δ=7.98 (d, ³J_{H,H}=9.2 Hz, 1H, 5-H), 6.12 (dd, ³J_{H,H}=9.2 Hz, ⁴J_{H,H}=2.3 Hz, 1H, 6-H), 6.04 (d, ⁴J_{H,H}=2.3 Hz, 1H, 2-H), 4.44 (br. s, 1H, NH), 3.93 (s, 3H, OCH₃), 3.18 (t, ³J_{H,H}=6.9 Hz, 2H, 1′-H₂), 1.65 (tt, ³J_{H,H}=7.3 Hz, ³J_{H,H}=7.3 Hz, 2H, 2′-H₂), 1.25-1.48 (m, 6H, 5′-H₂, 4′-H₂, 3′-H₂), 0.90 (br. t, ³J_{H,H}=6.9 Hz, 3H, 6′-H₃). MS (ESI): m/z (%)=253 (10) [M+H]⁺, 275 (33) [M+Na]⁺, 527 (100) [2 M+Na]. HRMS (ESI): calculated: 275.137162. found: 275.139537 [M+Na]⁺.

### Step 2

### N¹-hexyl-3-methoxybenzene-1,4-diamine

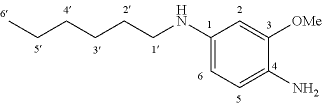 [SMILES: CCCCCCNc1ccc(N)c(OC)c1]

In analogy to example 1 step 2, N-hexyl-3-methoxy-4-nitroaniline (145 mg, 0.575 mmol) was converted with Pd/C (10% w/w, 13 mg) and hydrogen in ethyl acetate (10 ml) to yield the title compound. The light-sensitive raw product was used without further purification for the next synthesis step.

### Step 3

### methyl 3-(N-(4-(hexylamino)-2-methoxyphenyl)sulfamoyl)thiophene-2-carboxylate

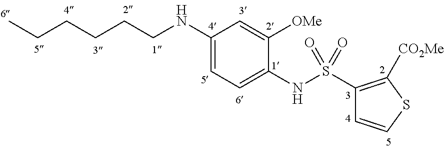 [SMILES: CCCCCCNc1ccc(NS(=O)(=O)c2ccsc2C(=O)OC)c(OC)c1]

In analogy to example 1 step 3, N¹-hexyl-3-methoxybenzene-1,4-diamine (total amount of the previous synthesis step), dimethylaminopyridine (35 mg, 0.29 mmol, 0.5 eq.) and 3-(chlorosulfonyl)-thiophene-2-carboxylic methyl ester (152 mg, 0.633 mmol, 1.1 eq.) were converted in pyridine (10 ml) to yield the title compound. After column chromatography purification (isohexane:ethyl acetate=2:1), this compound was obtained as yellow solid matter (175 mg, 71% over two steps). ¹H-NMR (399.788 MHz, CDCl₃): δ=8.12 (s, 1H, SO₂NH), 7.38 (d, ³J_{H,H}=5.0 Hz, 1H, 5-H), 7.35 (d, ³J_{H,H}=5.0 Hz, 1H, 4-H), 7.29 (d, ³J_{H,H}=8.7 Hz, 1H, 6′-H), 6.15 (dd, ³J_{H,H}=8.6 Hz, ⁴J_{H,H}=2.5 Hz, 1H, 5′-H), 5.95 (d, ⁴J_{H,H}=2.5 Hz, 1H, 3′-H), 4.00 (s, 3H, CO₂CH₃), 3.49 (s, 3H, OCH₃), 3.02 (t, ³J_{H,H}=7.2 Hz, 2H, 1″-H₂), 1.57 (tt, ³J_{H,H}=7.3 Hz, ³J_{H,H}=7.3 Hz, 2H, 2″-H), 1.24-1.39 (m, 6H, 3″-H₂, 4″-H₂, 5″-H₂), 0.88 (t, ³J_{H,H}=6.9 Hz, 3H, 6″-H₃). MS (ESI): m/z (%)=427 (55) [M+H]⁺, 449 (100) [M+Na]⁺. HRMS (ESI): calculated: 449.118086. found: 449.118829 [M+Na]⁺. CHN: calculated: 53.50% C, 6.14% H, 6.57% N. found: 53.71% C, 6.21% H, 6.65% N.

### Example 3

### Compound 2

### methyl 3-(N-(4-(hexylamino)-3-methoxyphenyl)sulfamoyl)thiophene-2-carboxylate

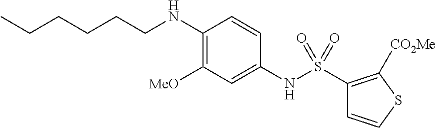 [SMILES: CCCCCCNc1ccc(NS(=O)(=O)c2ccsc2C(=O)OC)cc1OC]

### Step 1

### N-hexyl-2-methoxy-4-nitroaniline

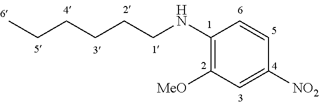 [SMILES: CCCCCCNc1ccc([N+](=O)[O-])cc1OC]

2-Methoxy-4-nitroaniline (1.01 g, 6.00 mmol, 1.2 eq.) and solid KOH (351 mg, 6.26 mmol, 1.3 eq.) was dissolved in a mixture of ^{t}BuOH (3.8 ml) and DMSO (1.3 ml), prior to the addition of hexyl iodide (740 μl, 1.06 g, 5.00 mmol). The reaction mixture was stirred for two hours under reflux and cooled to room temperature, followed by addition of ethyl acetate and washing with H₂O. The aqueous phase was then extracted with ethyl acetate and the combined organic phases were washed with saturated aqueous NaCl solution, dried over Na₂SO₄, filtered, and the solvent was removed under reduced pressure. Column chromatography purification (isohexane:ethyl acetate=5:1) yielded the title compound (1.03 g, 82%) as yellow solid matter. ¹H-NMR (399.788 MHz, CDCl₃): δ=7.98 (dd, ³J_{H,H}=8.8 Hz, ⁴J_{H,H}=2.3 Hz, 1H, 5-H), 7.61 (d, ⁴J_{H,H}=2.3 Hz, 1H, 3-H), 6.48 (d, ³J_{H,H}=8.9 Hz, 1H, 6-H), 5.01 (br. s, 1H, NH), 3.93 (s, 3H, OCH₃), 3.20-3.25 (m, 2H, 1′-H₂), 1.68 (tt, ³J_{H,H}=7.3 Hz, =7.3 Hz, 2H, 2′-H₂), 1.25-1.45 (m, 6H, 3′-H₂, 4′-H₂, 5′-H₂), 0.90 (br. t, ³J_{H,H}=7.0 Hz, 3H, 6′-H₃). MS (ESI): m/z (%)=253 (5) [M+H]⁺, 275 (6) [M+Na]⁺, 381 (100) [2 M−C₂H₆O₅N₂]⁺, 779 (17) [2 M+Na]⁺. HRMS (ESI): calculated: 275.137162. found: 275.134622 [M+Na]⁺.

### Step 2

### N¹-hexyl-2-methoxybenzene-1,4-diamine

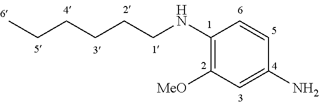 [SMILES: CCCCCCNc1ccc(N)cc1OC]

N-Hexyl-2-methoxy-4-nitroaniline (704 mg, 2.79 mmol) was dissolved in methanol and Pd/C (10% w/w) was added. Subsequently, the gas phase was removed several times by evacuation and replaced by hydrogen. The reaction mixture was stirred at room temperature, filtered through silica gel and the solvent was removed under reduced pressure, yielding the raw product as violet solid matter (606 mg, ≈98%). ¹H-NMR (399.788 MHz, CDCl₃): δ=6.49 (d, ³J_{H,H}=8.9 Hz, 1H, 6-H), 6.31-6.24 (m, 2H, 3-H, 5-H), 3.80 (s, 3H, OCH₃), 3.44 (br. s, 3H, NH, NH₂), 3.04 (t, =7.1 Hz, 2H, 1′-H₂), 1.62 (quint, ³J_{H,H}=7.1 Hz, 2H, 2′-H₂), 1.45-1.36 (m, 2H, 5′-H₂), 1.35-1.26 (m, 4H, 3′-H₂, 4′-H₂), 0.93-0.86 (m, 3H, 6′-H₃). MS (EI): m/z (%)=223 (19), 222 (100) [M]⁺, 207 (13), 152 (13), 151 (86), 136 (27). HRMS (EI): calculated: 222.1732. found: 222.1723.

### Step 3

### methyl 3-(N-(4-(hexylamino)-3-methoxyphenyl)sulfamoyl)thiophene-2-carboxylate

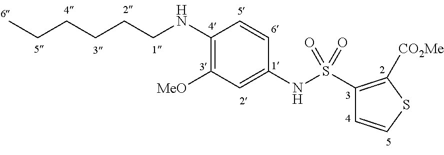 [SMILES: CCCCCCNc1ccc(NS(=O)(=O)c2ccsc2C(=O)OC)cc1OC]

N¹-Hexyl-2-methoxyphenyl-1,4-diamine (500 mg, 2.25 mmol) was dissolved in dichloromethane (6.75 ml), followed by addition of triethylamine (0.38 ml, 2.25 mmol, 1.0 eq.) and a solution of 3-(chlorosulfonyl)-thiophene-2-carboxylic methyl ester (541 mg, 2.25 mmol, 1.0 eq.) in dichloromethane at 0° C. The reaction mixture was slowly heated to room temperature and stirred for approx. 16 h. The reaction mixture was subsequently washed with saturated aqueous ammonium chloride solution and the aqueous phase was extracted with dichloromethane. The combined organic phases were washed with saturated aqueous NaCl solution, dried over Na₂SO₄ and the solvent was removed under reduced pressure. Column chromatography purification (dichloromethane) yielded the title compound (220 mg, 23%) as orange-colored viscous liquid. ¹H-NMR (399.788 MHz, CDCl₃): δ=7.96 (s, 1H, SO₂NH), 7.42 (d, ³J_{H,H}=5.3 Hz, 1H, 5-H), 7.38 (d, ³J_{H,H}=5.0 Hz, 1H, 4-H), 6.68 (d, ⁴J_{H,H}, =2.1 Hz, 1H, 2′-H), 6.42 (dd, ³J_{H,H}=8.3 Hz, ⁴J_{H,H}=2.3 Hz, 1H, 6′-H), 6.33 (d, ³J_{H,H}=8.3 Hz, 1H, 5′-H), 4.08 (br. s, 1H, NH), 4.01 (s, 3H, CO₂CH₃), 3.77 (s, 3H, OCH₃), 3.01 (t, ³J_{H,H}=7.1 Hz, 2H, 1″-H₂), 1.59 (quint, ³J_{H,H}=7.1 Hz, 2H, 2″-H₂), 1.42-1.33 (m, 2H, 5″-H₂), 1.32-1.23 (m, 4H, 3″-H₂, 4″-H₂), 0.91-0.86 (m, 3H, 6″-H₂). MS (EI): m/z (%)=427 (8), 426 (24) [M]⁺, 394 (5), 222 (22), 221 (100), 137 (6). (EI-HR): calculated: 426.1283. found: 426.1290. CHN: calculated: C: 53.50%, H: 6.14%, N: 6.57%. found: C: 52.41%, H: 6.187%, N: 6.60%.

### Example 4

### Compound 3

### methyl 3-(N-(4-(isopentylamino)-2-methoxyphenyl)sulfamoyl)thiophene-2-carboxylate

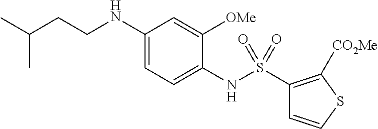 [SMILES: COC(=O)c1sccc1S(=O)(=O)Nc1ccc(NCCC(C)C)cc1OC]

### Step 1

### N-isopentyl-3-methoxy-4-nitroaniline

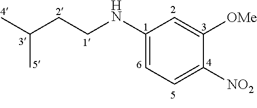 [SMILES: COc1cc(NCCC(C)C)ccc1[N+](=O)[O-]]

In analogy to example 1 step 1,4-fluoro-2-methoxy-1-nitrobenzene (171 mg, 1.00 mmol), isopentylamine (174 mg, 2.00 mmol, 2.0 eq.) and triethylamine (0.56 ml, 405 mg, 4.00 mmol, 4.0 eq.) were dissolved in acetonitrile (3 ml) and heated for one hour in a sealed reaction vial in a microwave reactor to 120° C. The raw product was adsorbed to silica gel and purified using column chromatography purification (cycloxane-ethyl acetate gradient) to yield the title compound (187 mg, 78%, yellow solid matter). ¹H-NMR (399.788 MHz, CDCl₃): δ=7.98 (d, ³J_{H,H}=9.2 Hz, 1H, 5-H), 6.14 (dd, ³J_{H,H}=9.2 Hz, ³J_{H,H}=2.3 Hz, 1H, 6-H), 6.06 (d, ³J_{H,H}=2.3 Hz, 1H, 2-H), 3.92 (s, 3H, OCH₃), 3.20 (t, ³J_{H,H}=7.4 Hz, 2H, 1′-H₂), 1.72 (tsep, ³J_{H,H}=6.6 Hz, ³J_{H,H}=6.6 Hz, 1H, 3′-H), 1.54 (dt, ³J_{H,H}=7.3 Hz, ³J_{H,H}=7.3 Hz, 2H, 2′-H₂), 0.96 (d, ³J_{H,H}=6.6 Hz, 6H, 4′-H₃, 5′-H₃). MS (ESI): m/z (%)=239 (76) [M+H]⁺, 261 (35) [M+Na]⁺, 499 (100) [2 M+Na]⁺, 737 (22) [3 M+Na]⁺. HRMS (ESI): calculated: 239.139568. found: 239.135143 [M+H]⁺.

### Step 2

### N¹-isopentyl-3-methoxybenzene-1,4-diamine

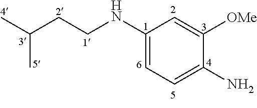

In analogy to example 1 step 2, N-isopentyl-3-methoxy-4-nitroaniline (238 mg, 1.00 mmol) was converted with Pd/C (10% w/w, 26 mg) and hydrogen in ethyl acetate (7 ml) to yield the title compound. The light-sensitive raw product was used without further purification for the next synthesis step.

### Step 3

### methyl 3-(N-(4-(isopentylamino)-2-methoxyphenyl)sulfamoyl)thiophene-2-carboxylate

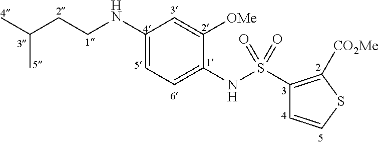 [SMILES: COC(=O)c1sccc1S(=O)(=O)Nc1ccc(NCCC(C)C)cc1OC]

In analogy to example 1 step 3, N¹-isopentyl-3-methoxyphenyl-1,4-diamine (total amount of the previous synthesis step) was converted with dimethylaminopyridine (61 mg, 0.50 mmol, 0.5 eq.) and 3-(chlorosulfonyl)-thiophene-2-carboxylic methyl ester (289 mg, 1.20 mmol, 1.2 eq.) in pyridine (10 ml) to yield the title compound. After column chromatography purification (isohexane:ethyl acetate=2:1), this compound was obtained as yellow solid matter (173 mg, 42% over two steps). ¹H-NMR (399.788 MHz, CDCl₃): δ=8.13 (s, 1H, SO₂NH), 7.39 (d, ³J_{H,H}=5.3 Hz, 1H, 5-H), 7.36 (d, ³J_{H,H}=5.3 Hz, 1H, 4-H), 7.30 (d, ³J_{H,H}=8.5 Hz, 1H, 6′-H), 6.18 (dd, ³J_{H,H}=8.3 Hz, ⁴J_{H,H}=2.1 Hz, 1H, 5′-H), 5.98 (d, ³J_{H,H}=2.1 Hz, 1H, 3′-H), 4.00 (s, 3H, CO₂CH₃), 3.50 (s, 3H, OCH₃), 3.04 (t, ³J_{H,H}=7.4 Hz, 1H, 1″-H), 1.67 (sep, ³J_{H,H}=6.6 Hz, 1H, 3″-H), 1.45-1.50 (m, 2H, 2″-H₂), 0.92 (d, ³J_{H,H}=6.6 Hz, 6H, 4″-H₃, 5″-H₃). MS (ESI): m/z (%)=208 (68) [M+C₆H₄O₄S₂]⁺, 413 (100) [M+H]⁺, 435 (41) [M+Na]⁺, 825 (31) [2 M+H]⁺, 847 (32) [2 M+Na]⁺. HRMS (ESI): calculated: 413.120491. found: 413.123613 [M+H]⁺. CHN: calculated: 52.41% C, 5.86% H, 6.79% N. found: 52.48% C, 5.99% H, 6.61% N.

### Example 5

### Compound 4

### methyl 3-(N-(4-(benzylamino)-2-methoxyphenyl)sulfamoyl)thiophene-2-carboxylate

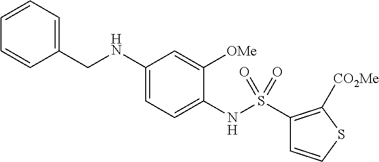 [SMILES: COC(=O)c1sccc1S(=O)(=O)Nc1ccc(NCc2ccccc2)cc1OC]

### Step 1

### N-benzyl-3-methoxy-4-nitroaniline

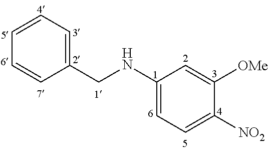 [SMILES: COc1cc(NCc2ccccc2)ccc1[N+](=O)[O-]]

In analogy to example 2 step 1,4-fluoro-2-methoxy-1-nitrobenzene (2.00 g, 11.7 mmol) was converted with benzylamine (3.19 ml, 3.13 g, 29.2 mmol, 2.5 eq.) and K₂CO₃ (2.42 g, 17.5 mmol, 1.5 eq.) in acetonitrile (130 ml) to yield the title compound (2.39 g, 79%, yellow solid matter). ¹H-NMR (399.788 MHz, CDCl₃): δ=7.98 (d, ³J_{H,H}=8.9 Hz, 1H, 5-H), 7.30-7.40 (m, 5H, Ar—H) 6.18 (dd, ³J_{H,H}=9.2 Hz, ⁴J_{H,H}=2.3 Hz, 1H, 6-H), 6.09 (d, ⁴J_{H,H}=2.3 Hz, 1H, 2-H), 4.82 (br. s, 1H, NH), 4.42 (d, ³J_{H,H}=5.5 Hz, 2H, 1′-H₂), 3.86 (s, 3H, OCH₃). MS (ESI): m/z (%)=259 (100) [M+H]⁺, 517 (58) [2 M+H]⁺, 775 (65) [3 M+H]⁺. HRMS (ESI): calculated: 259.10772. found: 259.108268 [M+H]⁺.

### Step 2

### N¹-benzyl-3-methoxybenzene-1,4-diamine

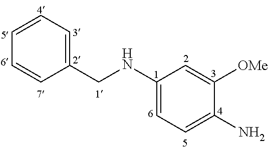 [SMILES: COc1cc(NCc2ccccc2)ccc1N]

N-Benzyl-3-methoxy-4-nitroaniline (272 mg, 1.00 mmol) and tin(II)chloride dihydrate (1.13 g, 5.00 mmol, 5.0 eq.) was dissolved in ethyl acetate and heated for 24 h under reflux. Subsequently, ethyl acetate (100 ml) and saturated aqueous NaHCO₃ solution (100 ml) was added and the resulting solution was filtered through silica gel. The organic phase was washed with saturated aqueous NaHCO₃ solution (2×100 ml) and saturated aqueous NaCl solution, dried over Na₂SO₄, filtered, and the solvent was removed under reduced pressure. The light-sensitive raw product was obtained as brown-black liquid and used without further purification for the next synthesis step.

### Step 3

### methyl 3-(N-(4-(benzylamino)-2-methoxyphenyl)sulfamoyl)thiophene-2-carboxylate

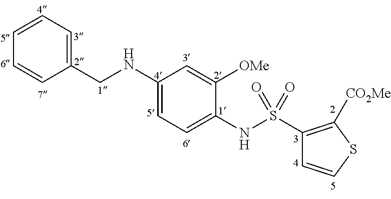 [SMILES: COC(=O)c1sccc1S(=O)(=O)Nc1ccc(NCc2ccccc2)cc1OC]

In analogy to example 1 step 3, N¹-benzyl-3-methoxyphenyl-1,4-diamine (total amount of the previous synthesis step) was converted with dimethylaminopyridine (61 mg, 0.50 mmol, 0.5 eq.) and 3-(chlorosulfonyl)-thiophene-2-carboxylic methyl ester (264 mg, 1.10 mmol, 1.1 eq.) in pyridine (10 ml) to yield the title compound. After column chromatography purification (isohexane:ethyl acetate=gradient from 6:1 to 2:1), the compound was obtained as yellow solid matter (145 mg, 34% over two steps). ¹H-NMR (399.788 MHz, CDCl₃): δ=8.06 (s, 1H, SO₂NH), 7.32 (d, 5.3 Hz, 1H, 5-H), 7.30 (d, ³J_{H,H}=5.3 Hz, 1H, 4-H), 7.23 (d, ³J_{H,H}=8.7 Hz, 1H, 6′-H), 7.19-7.27 (m, 5H, 3″-H, 4″-H, 5″-H, 6″-H, 7″-H), 6.11 (dd, ³J_{H,H}=8.6 Hz, ⁴J_{H,H}=2.4 Hz, 1H, 5′-H), 5.91 (d, ⁴J_{H,H}=2.5 Hz, 1H, 3′-H), 4.19 (s, 2H, 1″-H₂), 3.96 (br. s, 1H, NH), 3.94 (s, 3H, CO₂CH₃), 3.39 (s, 3H, OCH₃). MS (ESI): m/z (%)=455 (82) [M+Na]⁺, 887 (100) [2 M+Na]⁺, 1319 (22) [3 M+Na]⁺. HRMS (ESI): calculated: 455.071135. found: 455.067108 [M+Na]⁺. CHN: calculated: 55.45% C, 4.66% H, 6.48% N. found: 55.59% C, 4.80% H, 6.39% N.

### Example 6

### Compound 5

### methyl 3-(N-(4-(tert-butylamino)-2-methoxyphenyl)sulfamoyl)thiophene-2-carboxylate

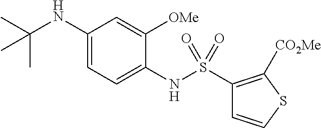 [SMILES: COC(=O)c1sccc1S(=O)(=O)Nc1ccc(NC(C)(C)C)cc1OC]

### Step 1

### N-(tert-butyl)-3-methoxy-4-nitroaniline

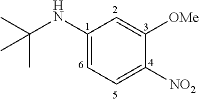 [SMILES: COc1cc(NC(C)(C)C)ccc1[N+](=O)[O-]]

In analogy to example 1 step 1,4-fluoro-2-methoxy-1-nitrobenzene (516 mg, 3.02 mmol) was converted with tert-butylamine (1.10 g, 15.1 mmol, 5.0 eq.) in acetonitrile (4 ml) for three hours in a sealed reaction vial in a microwave reactor to yield the title compound (169 mg, 25%, yellow solid matter). ¹H-NMR (399.788 MHz, CDCl₃): δ=7.95 (d, ³J_{H,H}=9.2 Hz, 1H, 5-H), 6.21 (br. d, ³J_{H,H}=9.2 Hz, 1H, 6-H), 6.15 (br. s, 1H, 2-H), 4.53 (br. s, 1H, NH), 3.91 (s, 3H, OCH₃), 1.44 (s, 9H, C(CH₃)₃). MS (ESI): m/z (%)=225 (100) [M+H]⁺, 449 (12) [2 M+H]⁺, 673 (9) [3 M+H]⁺. HRMS (ESI): calculated: 225.123918. found: 225.121415 [M+H]⁺.

### Step 2

### N¹-(tert-butyl)-3-methoxybenzene-1,4-diamine

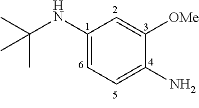

In analogy to example 1 step 2, N-tert-butyl-3-methoxy-4-nitroaniline (163 mg, 0.727 mmol) was converted with Pd/C (10% w/w, 19 mg) and hydrogen in ethyl acetate (5 ml) to yield the title compound. The light-sensitive raw product was used without further purification for the next synthesis step.

### Step 3

### methyl 3-(N-(4-(tert-butylamino)-2-methoxyphenyl)sulfamoyl)thiophene-2-carboxylate

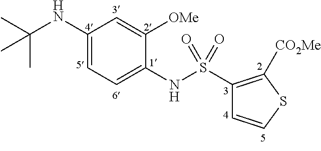 [SMILES: COC(=O)c1sccc1S(=O)(=O)Nc1ccc(NC(C)(C)C)cc1OC]

In analogy to example 1 step 3, N¹-tert-butyl-3-methoxyphenyl-1,4-diamine (total amount of the previous synthesis step) was converted with dimethylaminopyridine (44 mg, 0.36 mmol, 0.5 eq.) and 3-(chlorosulfonyl)-thiophene-2-carboxylic methyl ester (192 mg, 0.8 mmol, 1.1 eq.) in pyridine (10 ml) to yield the title compound. After column chromatography purification (isohexane:ethyl acetate=2:1), this compound was obtained as yellow solid matter (231 mg, 80% over two steps). ¹H-NMR (399.788 MHz, DMSO-d₆): δ=8.39 (s, 1H, SO₂NH), 7.87 (d, ³J_{H,H}=5.3 Hz, 1H, 5-H), 7.29 (d, ³J_{H,H}=5.3 Hz, 1H, 4-H), 6.93 (d, ³J_{H,H}=8.5 Hz, 1H, 6′-H), 6.20 (dd, ³J_{H,H}=8.7, ⁴J_{H,H}=2.3 Hz, 1H, 5′-H), 6.17 (d, ⁴J_{H,H}=2.3 Hz, 1H, 3′-H), 5.23 (br. s, 1H, NH), 3.93 (s, 3H, CO₂CH₃), 3.40 (s, 3H, OCH₃), 1.25 (s, 9H, C(CH₃)₃). MS (ESI): m/z (%)=163 (14) [M−C₆H₄O₄S₂]⁺, 399 (100) [M+H]⁺, 421 (18) [M+Na], 797 (22) [2 M+H]⁺. HRMS (EI): calculated: 398.097016. found: 398.100247 [M]⁺. CHN: calculated: 51.24% C, 5.56% H, 7.03% N. found: 51.47% C, 5.60% H, 6.94% N.

### Example 7

### Compound 6

### methyl 3-(N-(2-methoxy-4-(propylamino)phenyl)sulfamoyl)thiophene-2-carboxylate

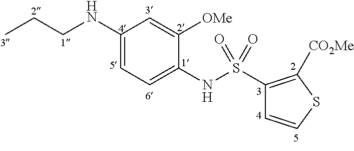 [SMILES: CCCNc1ccc(NS(=O)(=O)c2ccsc2C(=O)OC)c(OC)c1]

¹H-NMR (399.788 MHz, CDCl₃): δ=8.09 (br. s, 1H, SO₂NH), 7.38 (d, ³J_{H,H}=5.3 Hz, 1H, 5-H), 7.35 (d, ³J_{H,H}=5.3 Hz, 1H, 4-H), 7.28 (d, ³J_{H,H}=8.7 Hz, 1H, 6′-H), 6.12 (dd, ³J_{H,H}=8.7 Hz, ⁴J_{H,H}=2.5 Hz, 1H, 5′-H), 5.92 (d, ⁴J_{H,H}=2.5 Hz, 1H, 3′-H), 4.00 (s, 3H, CO₂CH₃), 3.59 (br. s, 1H, NH), 3.48 (s, 3H, OCH₃), 3.00 (t, ³J_{H,H}=7.1 Hz, 2H, 1″-H₂), 1.60 (tq, ³J_{H,H}=7.1 Hz, ³J_{H,H}=7.1 Hz, 2H, 2″-H₂), 0.97 (t, ³J_{H,H}=7.3 Hz, 3H, 3″-H₃). MS (EI): m/z (%)=179 (100) [M−C₆H₆O₄S₂]⁺, 384 (19) [M]⁺. MS (ESI): m/z (%)=385 (17) [M+H]⁺, 769 (100) [2 M+H]⁺, 791 (33) [2 M+Na], 1153 (28) [3 M+H]⁺, 1176 (13) [3 M+Na]. HRMS (EI): calculated: 384.081366. found: 384.083550 [M]⁺.

### Example 8

### Compound 7

### methyl 3-(N-(4-(butylamino)-2-methoxyphenyl)sulfamoyl)thiophene-2-carboxylate

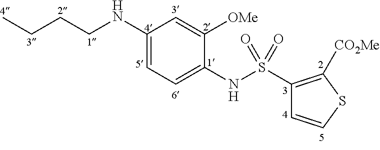 [SMILES: CCCCNc1ccc(NS(=O)(=O)c2ccsc2C(=O)OC)c(OC)c1]

¹H-NMR (399.788 MHz, CDCl₃): δ=8.09 (s, 1H, SO₂NH), 7.38 (d, ³J_{H,H}=5.3 Hz, 1H, 5-H), 7.35 (d, ³J_{H,H}=5.3 Hz, 1H, 4-H), 7.28 (d, ³J_{H,H}=8.7 Hz, 1H, 6′-H), 6.12 (dd, ³J_{H,H}=8.7 Hz, ⁴J_{H,H}=2.5 Hz, 1H, 5′-H), 5.92 (d, ³J_{H,H}=2.3 Hz, 1H, 3′-H), 4.00 (s, 3H, CO₂CH₃), 3.59 (br. s, 1H, NH), 3.48 (s, 3H, OCH₃), 3.03 (t, ³J_{H,H}=7.1 Hz, 2H, 1″-H₂), 1.52-1.59 (m, 2H, 2″-H₂), 1.35-1.44 (m, 2H, 3″-H₂), 0.93 (t, ³J_{H,H}=7.3 Hz, 3H, 4″-H₃). MS (ESI): m/z (%)=399 (6) [M+H]⁺, 421 (50) [M+Na]⁺, 819 (100) [2 M+Na]⁺, 1217 (26) [3 M+Na]. HRMS (ESI): calculated: 421.086785. found: 421.083904 [M+Na]⁺.

### Example 9

### Compound 8

### methyl 3-(N-(2-methoxy-4-(pentylamino)phenyl)sulfamoyl)thiophene-2-carboxylate

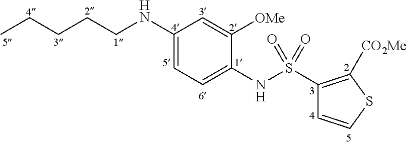 [SMILES: CCCCCNc1ccc(NS(=O)(=O)c2ccsc2C(=O)OC)c(OC)c1]

¹H-NMR (399.788 MHz, CDCl₃): δ=8.09 (br. s, 1H, SO₂NH), 7.38 (d, ³J_{H,H}=5.3 Hz, 1H, 5-H), 7.35 (d, ³J_{H,H}=5.3 Hz, 1H, 4-H), 7.28 (d, ³J_{H,H}=8.7 Hz, 1H, 6′-H), 6.12 (dd, ³J_{H,H}=8.6 Hz, ⁴J_{H,H}=2.5 Hz, 1H, 5′-H), 5.92 (d, ⁴J_{H,H}=2.5 Hz, 1H, 3′-H), 4.00 (s, 3H, CO₂CH₃), 3.59 (br. s, 1H, NH), 3.48 (s, 3H, OCH₃), 3.02 (t, ³J_{H,H}=7.1 Hz, 2H, 1″-H₂), 1.54-1.61 (m, 2H, 2″-H₂), 1.31-1.37 (m, 4H, 3″-H₂, 4″-H₂), 0.90 (t, ³J_{H,H}=7.1 Hz, 3H, 5″-H₃). MS (ESI): m/z (%)=413 (100) [M+H]⁺, 430 (70) [M+NH₄]⁺, 435 (20) [M+Na]⁺, 825 (42) [2 M+H]⁺. HRMS (EI): calculated: 412.112666. found: 412.111911 [M]⁺.

### Example 10

### Compound 9

### methyl 3-(N-(4-(isopropylamino)-2-methoxyphenyl)sulfamoyl)thiophene-2-carboxylate

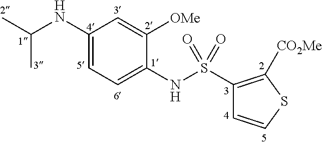 [SMILES: COC(=O)c1sccc1S(=O)(=O)Nc1ccc(NC(C)C)cc1OC]

¹H-NMR (399.788 MHz, CDCl₃): δ=8.10 (s, 1H, SO₂NH), 7.38 (d, ³J_{H,H}=5.3 Hz, 1H, 5-H), 7.36 (d, ³J_{H,H}=5.3 Hz, 1H, 4-H), 7.28 (d, ³J_{H,H}=8.5 Hz, 1H, 6′-H), 6.14 (dd, ³J_{H,H}=8.6 Hz, ⁴J_{H,H}=2.3 Hz, 1H, 5′-H), 5.94 (d, ³J_{H,H}=2.3 Hz, 1H, 3′-H), 4.00 (s, 3H, CO₂CH₃), 3.53 (sep, ³J_{H,H}=6.4 Hz, 1H, 1″-H), 3.48 (s, 3H, OCH₃), 1.17 (d, ³J_{H,H}=6.4 Hz, 6H, 2″-H₆). MS (ESI): m/z (%)=385 (21) [M+H]⁺, 769 (100) [2 M+H]⁺, 1154 (16) [3 M+2H]⁺. HRMS (ESI): calculated: 385.089191. found: 385.092388 [M+H]⁺.

### Example 11

### Compound 10

### methyl 3-(N-(4-(isobutylamino)-2-methoxyphenyl)sulfamoyl)thiophene-2-carboxylate

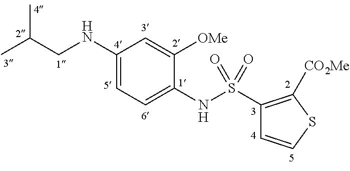 [SMILES: COC(=O)c1sccc1S(=O)(=O)Nc1ccc(NCC(C)C)cc1OC]

¹H-NMR (399.788 MHz, DMSO-d₆): δ=8.35 (s, 1H, SO₂NH), 7.86 (d, ³J_{H,H}=5.0 Hz, 1H, 5-H), 7.26 (d, ³J_{H,H}=5.0 Hz, 1H, 4-H), 6.94 (m_{c}, 1H, 6′-H), 6.03-6.06 (m, 2H, 3′-H, 5′-H), 5.65 (t, ³J_{H,H}=5.6 Hz, 1H, NH), 3.93 (s, 3H, CO₂CH₃), 3.41 (s, 3H, OCH₃), 2.75 (dd, ³J_{H,H}=6.2 Hz, ³J_{H,H}=6.2 Hz, 1H, 1″-H), 1.77 (sep, ³J_{H,H}=6.6 Hz, 1H, 2″-H), 0.90 (d, ³J_{H,H}=6.6 Hz, 6H, 3″-H₃, 4″-H₃). MS (ESI): m/z (%)=194 (84) [M−C₆H₄O₄S₂]⁺, 399 (100) [M+H]⁺, 421 (63) [M+Na]⁺, 797 (30) [2 M+H]⁺, 819 (40) [2 M+Na]⁺. HRMS (EI): calculated: 398.097016. found: 398.100771 [M]⁺.

### Example 12

### Compound 11

### methyl 3-(N-(2-methoxy-4-((4-methylpentyl)amino)phenyl)sulfamoyl)thiophene-2-carboxylate

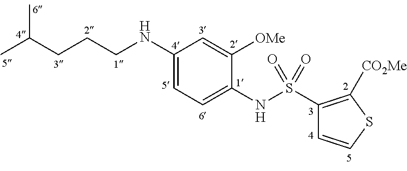 [SMILES: COC(=O)c1sccc1S(=O)(=O)Nc1ccc(NCCCC(C)C)cc1OC]

¹H-NMR (399.788 MHz, DMSO-d₆): δ=8.36 (br. s, 1H, SO₂NH), 7.86 (d, ³J_{H,H}=5.3 Hz, 1H, 5-H), 7.26 (d, ³J_{H,H}=5.0 Hz, 1H, 4-H), 6.94 (m, 1H, 6′-H), 6.02-6.05 (m, 2H, 5′-H, 3′-H), 5.59 (t, ³J_{H,H}=5.4 Hz, 1H, NH), 3.93 (s, 3H, CO₂CH₃), 3.41 (s, 3H, OCH₃), 2.91 (m_{c}, 2H, 1″-H₂), 1.45-1.58 (m, 3H, 4″-H, 2″-H₂), 1.20-1.26 (m, 2H, 3″-H₂), 0.85 (d, ³J_{H,H}=6.6 Hz, 6H, 5″-H₃, 6″-H₃). MS (ESI): m/z (%)=222 (71) [M−C₆H₄O₄S₂]⁺, 427 (100) [M+H]⁺, 449 (58) [M+Na], 854 (29) [2 M+H]⁺, 876 (36) [2 M+Na]⁺. HRMS (EI): calculated: 426.128316. found: 426.129707 [M]⁺.

### Example 13

### Compound 12

### methyl 3-(N-(2-methoxy-4-((5-methylhexyl)amino)phenyl)sulfamoyl)thiophene-2-carboxylate

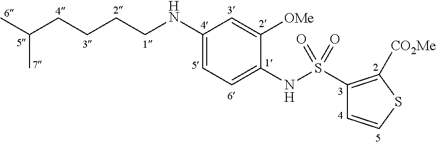 [SMILES: COC(=O)c1sccc1S(=O)(=O)Nc1ccc(NCCCCC(C)C)cc1OC]

¹H-NMR (399.788 MHz, DMSO-d₆): δ=8.36 (br. s, 1H, SO₂NH), 7.86 (d, ³J_{H,H}=5.3 Hz, 1H, 5-H), 7.26 (d, ³J_{H,H}=5.0 Hz, 1H, 4-H), 6.94 (m, 1H, 6′-H), 6.03-6.05 (m, 2H, 5′-H, 3′-H), 5.59 (t, ³J_{H,H}=5.3 Hz, 1H, NH), 3.93 (s, 3H, CO₂CH₃), 3.41 (s, 3H, OCH₃), 2.92 (m_{c}, 2H, 1″-H₂), 1.44-1.57 (m, 3H, 5″-H, 2″-H₂), 1.28-1.36 (m, 2H, 3″-H₂), 1.14-1.19 (m, 2H, 4″-H₂), 0.85 (d, ³J_{H,H}=6.6 Hz, 6H, 6″-H₃, 7″-H₃). MS (ESI): m/z (%)=236 (93) [M−C₆H₄O₄S₂]⁺, 441 (100) [M+H]⁺, 463 (61) [M+Na]⁺, 903 (33) [2 M+Na]⁺. HRMS (EI): calculated: 440.143966. found: 440.140167 [M]⁺.

### Example 14

### Compound 13

### methyl 3-(N-(4-(cyclohexylamino)-2-methoxyphenyl)sulfamoyl)thiophene-2-carboxylate

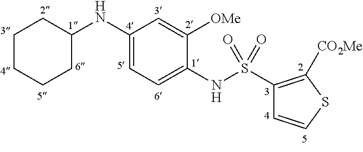 [SMILES: COC(=O)c1sccc1S(=O)(=O)Nc1ccc(NC2CCCCC2)cc1OC]

¹H-NMR (399.788 MHz, CDCl₃): δ=8.10 (br. s, 1H, SO₂NH), 7.38 (d, ³J_{H,H}=5.3 Hz, 1H, 5-H), 7.36 (d, ³J_{H,H}=5.3 Hz, 1H, 4-H), 7.27 (d, ³J_{H,H}=9.2 Hz, 1H, 6′-H), 6.15 (dd, ³J_{H,H}=8.5 Hz, ⁴J_{H,H}=1.8 Hz, 1H, 5′-H), 5.95 (br. s, 1H, 3′-H), 4.00 (s, 3H, CO₂CH₃), 3.48 (s, 3H, OCH₃), 3.15 (m_{c}, 1″-H), 1.99 (m_{c}, 2H, cyclohexyl-H), 1.73 (m_{c}, 2H, cyclohexyl-H), 1.63 (m_{c}, 1H, cyclohexyl-H), 1.07-1.38 (m, 5H, cyclohexyl-H). MS (EI): m/z (%)=111 (83) [C₅H₃OS]⁺, 219 (100) [M−C₆H₅O₄S₂]⁺, 220 (97) [M−C₁₃H₁₈NO]⁺, 424 (13) [M]⁺. HRMS (EI): calculated: 424.1127. found: 424.1138 [M]⁺.

### Example 15

### Compound 14

### methyl 3-(N-(2-ethoxy-4-(hexylamino)phenyl)sulfamoyl)thiophene-2-carboxylate

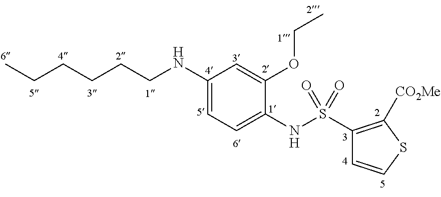 [SMILES: CCCCCCNc1ccc(NS(=O)(=O)c2ccsc2C(=O)OC)c(OCC)c1]

¹H-NMR (399.788 MHz, CDCl₃): δ=8.21 (br. s, 1H, SO₂NH), 7.38 (d, ³J_{H,H}=5.0 Hz, 1H, 5-H), 7.35 (d, ³J_{H,H}=5.3 Hz, 1H, 4-H), 7.32 (d, ³J_{H,H}=8.4 Hz, 1H, 6′-H), 6.13 (dd, ³J_{H,H}=8.6 Hz, ⁴J_{H,H}=2.4 Hz, 1H, 5′-H), 5.93 (d, ⁴J_{H,H}=2.3 Hz, 1H, 3′-H), 3.97 (s, 3H, CO₂CH₃), 3.76 (q, ³J_{H,H}=6.9 Hz, 2H, 1′″-H₂), 3.55 (br. s, 1H, NH), 3.02 (t, ³J_{H,H}=7.1 Hz, 2H, 1″-H₂), 1.56 (m_{c}, 2H, 2″-H₂), 1.28-1.40 (m, 6H, 3′-H₂, 4′-H₂, 5′-H₂), 0.85-0.90 (m, 6H, 6″-H₃, 2″-H₃). MS (ESI): m/z (%)=236 (100) [M−C₆H₄O₄S₂]⁺, 441 (38) [M+H]⁺, 463 (73) [M+Na], 479 (6) [M+K]⁺. HRMS (ESI) calculated: 441.151791. found: 441.153110 [M+H]⁺. HRMS (ESI): calculated: 463.133736. found: 463.136782 [M+Na]⁺.

### Example 16

### Compound 15

### methyl 3-(N-(4-(decylamino)-3-methoxyphenyl)sulfamoyl)thiophene-2-carboxylate

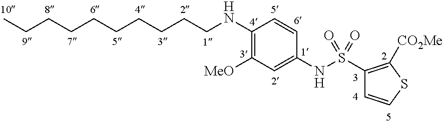 [SMILES: CCCCCCCCCCNc1ccc(NS(=O)(=O)c2ccsc2C(=O)OC)cc1OC]

¹H-NMR (399.788 MHz, CDCl₃): δ=7.96 (s, 1H, SO₂NH), 7.42 (d, ³J_{H,H}=5.3 Hz, 1H, 5-H), 7.38 (d, ³J_{H,H}=5.3 Hz, 1H, 4-H), 6.68 (d, ⁴J_{H,H}=2.2 Hz, 1H, 2′-H), 6.41 (dd, ³J_{H,H}=8.2 Hz, ⁴J_{H,H}=2.0 Hz, 1H, 6′-H), 6.33 (d, ³J_{H,H}=8.3 Hz, 1H, 4′-H), 4.01 (s, 3H, CO₂CH₃), 3.77 (s, 3H, OCH₃), 3.01 (t, ³J_{H,H}=7.1 Hz, 2H, 1″-H₂), 1.59 (quint, ³J_{H,H}=7.1 Hz, 2H, 2″-H₂), 1.40-1.32 (m, 2H, 9″-H₂), 1.32-1.20 (m, 12H, 3″-H₂, 4″-H₂, 5″-H₂, 6″-H₂, 7″-H₂, 8″-H₂), 0.87 (t, ³J_{H,H}=7.1 Hz, 3H, 10″-H₃). MS (EI): m/z (%)=482 (47) [M]⁺, 448 (32), 278 (29), 277 (100), 244 (37), 243 (95), 229 (16), 33 (34). HRMS (EI): calculated: 482.1909. found: 482.1914.

### Example 17

### Compound 16

### methyl 3-(N-(4-(hexylamino)phenyl)sulfamoyl)thiophene-2-carboxylate

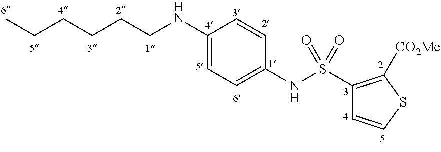 [SMILES: CCCCCCNc1ccc(NS(=O)(=O)c2ccsc2C(=O)OC)cc1]

¹H-NMR (399.788 MHz, CDCl₃): δ=7.90 (s, 1H, SO₂NH), 7.35 (d, ³J_{H,H}=5.3 Hz, 1H, 5-H), 7.30 (d, ³J_{H,H}=5.3 Hz, 1H, 4-H), 6.85-6.79 (m, 2H, 2′-H, 6′-H), 6.38-6.33 (m, 2H, 3′-H, 5′-H), 3.94 (s, 3H, CO₂CH₃), 2.94 (t, ³J_{H,H}=7.1 Hz, 2H, 1″-H₂), 1.49 (quint, ³J_{H,H}=7.1 Hz, 2H, 2″-H₂), 1.33-1.25 (m, 2H, 5″-H₂), 1.25-1.18 (m, 4H, 3″-H₂, 4″-H₂), 0.81 (t, ³J_{H,H}=6.6 Hz, 3H, 6″-H₃). MS (EI): m/z (%)=396 (30) [M]⁺, 192 (33), 191 (100), 121 (21), 111 (12). HRMS (EI): calculated: 396.1178. found: 396.1155.

### Example 18

### Compound 17

### methyl 3-(N-(4-(hexylamino)-2-propoxyphenyl)sulfamoyl)thiophene-2-carboxylate

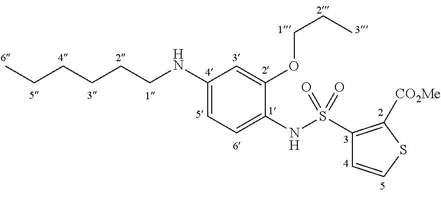 [SMILES: CCCCCCNc1ccc(NS(=O)(=O)c2ccsc2C(=O)OC)c(OCCC)c1]

¹H-NMR (399.788 MHz, CDCl₃): δ=8.20 (br. s, 1H, SO₂NH), 7.39 (d, ³J_{H,H}=5.0 Hz, 1H, 5-H), 7.36 (d, ³J_{H,H}=5.3 Hz, 1H, 4-H), 7.32 (d, ³J_{H,H}=8.7 Hz, 1H, 6′-H), 6.12 (dd, ³J_{H,H}=8.6 Hz, ⁴J_{H,H}=2.4 Hz, 1H, 5′-H), 5.94 (d, ⁴J_{H,H}=2.3 Hz, 1H, 3′-H), 3.97 (s, 3H, CO₂CH₃), 3.66 (t, ³J_{H,H}=6.6 Hz, 2H, 1′″-H₂) 3.55 (br. s, 1H, NH), 3.02 (t, ³J_{H,H}=7.1 Hz, 2H, 1″-H₂), 1.58 (m_{c}, 4H, 2″-H₂, 2″-H₂), 1.28-1.40 (m, 6H, 3″-H₂, 4″-H₂, 5″-H₂), 0.94 (t, ³J_{H,H}=7.4 Hz, 3H, 3″-H₃), 0.89 (br. t, ³J_{H,H}=7.0 Hz, 3H, 6″-H₃). MS (ESI): m/z (%)=250 (100) [M−C₆H₄O₄S₂]⁺, 455 (33) [M+H]⁺, 477 (60) [M+Na]. HRMS (ESI): calculated: 477.149386. found: 477.148384 [M+Na].

### Example 19

### Compound 18

### methyl 3-(N-(2-butoxy-4-(hexylamino)phenyl)sulfamoyl)thiophene-2-carboxylate

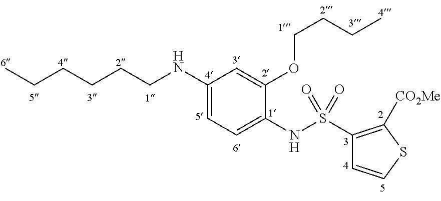 [SMILES: CCCCCCNc1ccc(NS(=O)(=O)c2ccsc2C(=O)OC)c(OCCCC)c1]

¹H-NMR (399.788 MHz, CDCl₃): δ=8.18 (br. s, 1H, SO₂NH), 7.38 (d, ³J_{H,H}=5.0 Hz, 1H, 5-H), 7.36 (d, ³J_{H,H}=5.3 Hz, 1H, 4-H), 7.32 (d, ³J_{H,H}=8.7 Hz, 1H, 6′-H), 6.12 (dd, ³J_{H,H}=8.7 Hz, ⁴J_{H,H}=2.5 Hz, 1H, 5′-H), 5.94 (d, ⁴J_{H,H}=2.3 Hz, 1H, 3′-H), 3.97 (s, 3H, CO₂CH₃), 3.69 (t, ³J_{H,H}=6.8 Hz, 2H, 1″-H₂), 3.51 (br. s, 1H, NH), 3.02 (t, ³J_{H,H}=7.1 Hz, 2H, 1″-H₂), 1.55 (m_{c}, 4H, 2″-H₂, 2″-H₂), 1.28-1.40 (m, 8H, 3′-H₂, 4″-H₂, 5″-H₂, 3″-H₂), 0.94 (t, ³J_{H H}=7.3 Hz, 3H, 5″-H₃), 0.89 (br. t, ³J_{H,H}=6.9 Hz, 3H, 6″-H₃). MS (ESI): m/z (%)=264 (100) [M−C₆H₄O₄S₂]⁺, 469 (15) [M+H]⁺, 491 (22) [M+Na]⁺. HRMS (ESI): calculated: 469.183091. found: 469.184613 [M+H]⁺.

### Example 20

### Compound 19

### methyl 3-(N-(4-(hexylamino)-2-(pentyloxy)phenyl)sulfamoyl)thiophene-2-carboxylate

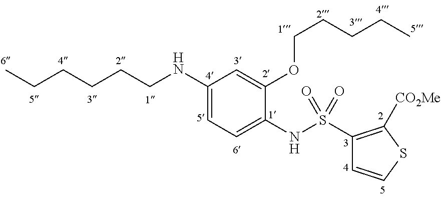 [SMILES: CCCCCCNc1ccc(NS(=O)(=O)c2ccsc2C(=O)OC)c(OCCCCC)c1]

¹H-NMR (399.788 MHz, CDCl₃): δ=8.18 (br. s, 1H, SO₂NH), 7.38 (d, ³J_{H,H}=5.3 Hz, 1H, 5-H), 7.36 (d, ³J_{H,H}=5.3 Hz, 1H, 4-H), 7.32 (d, ³J_{H,H}=8.7 Hz, 1H, 6′-H), 6.12 (dd, ³J_{H,H}=8.6 Hz, ⁴J_{H,H}=2.4 Hz, 1H, 5′-H), 5.94 (d, ⁴J_{H,H}=2.5 Hz, 1H, 3′-H), 3.97 (s, 3H, CO₂CH₃), 3.68 (t, ³J_{H,H}=7.0 Hz, 2H, 1′″-H₂), 3.54 (br. s, 1H, NH), 3.02 (t, ³J_{H,H}=7.2 Hz, 2H, 1″-H₂), 1.56 (m_{c}, 4H, 2″-H₂, 2″-H₂), 1.28-1.40 (m, 10H, 3′-H₂, 4″-H₂, 5″-H₂, 4″-H₂), 0.92 (t, ³J_{H,H}=7.1 Hz, 3H, 5″-H₃), 0.89 (br. t, ³J_{H,H}=7.1 Hz, 3H, 6″-H₃). MS (ESI): m/z (%)=278 (100) [M−C₆H₄O₄S₂]⁺, 483 (60) [M+H]⁺, 505 (58) [M+Na]⁺. HRMS (ESI): calculated: 483.198741. found: 483.201640 [M+H]⁺.

### Example 21

### Compound 20

### methyl 3-(N-(4-(hexylamino)-2-isopropoxyphenyl)sulfamoyl)thiophene-2-carboxylate

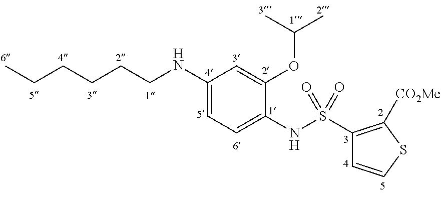 [SMILES: CCCCCCNc1ccc(NS(=O)(=O)c2ccsc2C(=O)OC)c(OC(C)C)c1]

¹H-NMR (399.788 MHz, CDCl₃): δ=8.21 (s, 1H, SO₂NH), 7.37 (d, ³J_{H,H}=5.3 Hz, 1H, 5-H), 7.35 (d, ³J_{H,H}=5.0 Hz, 1H, 4-H), 7.34 (d, ³J_{H,H}=8.7 Hz, 1H, 6′-H), 6.12 (dd, ³J_{H,H}=8.6 Hz, ⁴J_{H,H}=2.4 Hz, 1H, 5′-H), 5.93 (d, ⁴J_{H,H}=2.2 Hz, 1H, 3′-H) 4.35 (m_{c}, 1H, 1′″-H), 3.98 (s, 3H, CO₂CH₃), 3.53 (br. s, 1H, NH), 3.01 (t, ³J_{H,H}=7.1 Hz, 2H, 1″-H₂), 1.57 (m_{c}, 2H, 2″-H₂), 1.28-1.40 (m, 6H, 3″-H₂, 4″-H₂, 5″-H₂), 1.08 (d, ³J_{H,H}=6.0 Hz, 6H, 2″-H₃, 3″-H₃), 0.89 (t, ³J_{H,H}=6.9 Hz, 3H, 6″-H₃). MS (ESI): m/z (%)=250 (100) [M−C₆H₄O₄S₂]⁺, 455 (66) [M+H]⁺, 477 (68) [M+Na]⁺. HRMS (ESI): calculated: 455.167441. found: 455.164741 [M+H]⁺.

### Example 22

### Compound 21

### methyl 3-(N-(4-(hexylamino)-2-(isopentyloxy)phenyl)sulfamoyl)thiophene-2-carboxylate

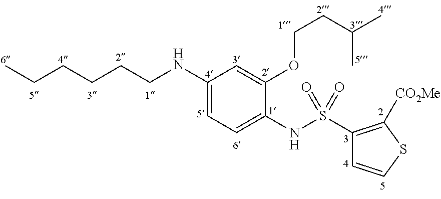 [SMILES: CCCCCCNc1ccc(NS(=O)(=O)c2ccsc2C(=O)OC)c(OCCC(C)C)c1]

¹H-NMR (399.788 MHz, CDCl₃): δ=8.16 (s, 1H, SO₂NH), 7.38 (d, ³J_{H,H}=5.3 Hz, 1H, 5-H), 7.36 (d, ³J_{H,H}=5.3 Hz, 1H, 4-H), 7.32 (d, ³J_{H,H}=8.5 Hz, 1H, 6′-H), 6.12 (dd, ³J_{H,H}=8.7 Hz, ⁴J_{H,H}=2.5 Hz, 1H, 5′-H), 5.95 (d, ⁴J_{H,H}=2.5 Hz, 1H, 3′-H), 3.98 (s, 3H, CO₂CH₃), 3.71 (t, ³J_{H,H}=7.1 Hz, 2H, 1′″-H₂), 3.66 (br. s, 1H, NH), 3.02 (t, ³J_{H,H}=7.1 Hz, 2H, 1″-H₂), 1.67 (m_{c}, 1H, 3′″-H), 1.57 (m_{c}, 2H, 2″-H₂), 1.25-1.40 (m, 8H, 3″-H₂, 4″-H₂, 5″-H₂, 2″-H₂), 0.92 (d, ³J_{H,H}=6.9 Hz, 6H, 3′″-H₃, 4′″-H₃), 0.89 (t, ³J_{H,H}=7.1 Hz, 3H, 6″-H₃). MS (ESI): m/z (%)=278 (100) [M−C₆H₄O₄S₂]⁺, 483 (62) [M+H]⁺, 505 (46) [M+Na]⁺. HRMS (ESI): calculated: 483.198741. found: 483.198026 [M+H]⁺.

### Example 23

### Compound 22

### methyl 3-(N-(4-(hexylamino)-2-phenethoxyphenyl)sulfamoyl)thiophene-2-carboxylate

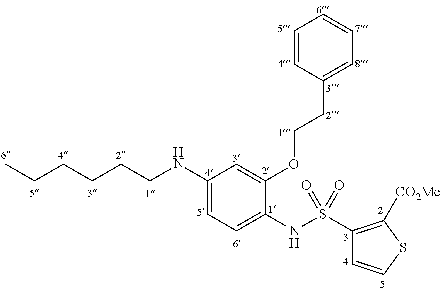 [SMILES: CCCCCCNc1ccc(NS(=O)(=O)c2ccsc2C(=O)OC)c(OCCc2ccccc2)c1]

¹H-NMR (399.788 MHz, CDCl₃): δ=8.19 (br. s, 1H, SO₂NH), 7.41 (d, ³J_{H,H}=5.3 Hz, 1H, 5-H), 7.39 (d, ³J_{H,H}=5.3 Hz, 1H, 4-H), 7.20-7.34 (m, 6H, 6′-H, 4″-H, 5′″-H, 6′″-H, 7′″-H, 8′″-H), 6.13 (dd, ³J_{H,H}=8.7 Hz, ⁴J_{H,H}=2.3 Hz, 1H, 5′-H), 5.92 (d, ⁴J_{H,H}=2.3 Hz, 1H, 3′-H), 3.97 (s, 3H, CO₂CH₃), 3.90 (t, ³J_{H,H}=7.4 Hz, 2H, 1′″-H₂), 3.53 (br. s, 1H, NH), 2.99 (t, ³J_{H,H}=7.1 Hz, 2H, 1″-H₂), 2.87 (t, ³J_{H,H}=7.7 Hz, 2H, 2″-H₂), 1.55 (m_{c}, 2H, 2″-H₂), 1.28-1.40 (m, 6H, 3″-H₂, 4″-H₂, 5″-H₂), 0.87 (br. t, ³J_{H,H}=6.9 Hz, 3H, 6″-H₃). MS (ESI): m/z (%)=312 (100) [M−C₆H₄O₄S₂]⁺, 517 (77) [M+H]⁺, 539 (56) [M+Na]⁺. HRMS (ESI): calculated: 517.183091. found: 517.181064 [M+H]⁺.

### Example 24

### Characterization of the Substances of this Invention

24.1 PPAR Beta/Delta Subtype-Specific Binding of the Substances of this Invention

A) Competitive ligand-binding of the substances of this invention was measured in vitro using time-resolved fluorescence resonance energy transfer (TR-FRET) by competition with the commercially available fluorescent Fluormone® Pan-PPAR-Green for binding to the fusion protein GST-PPAR beta/delta LBD (GST=glutathione S-transferase; LBD=ligand binding domain) (Invitrogen, Darmstadt, Germany) in a VICTOR3V Multilabel Counter (WALLAC 1420; PerkinElmer Life and Analytical Sciences, Rodgau, Germany). The measurement was performed in 100 mM KC, 20 mM Tris pH 7.9, 0.01% Triton X100 and 1 μg/μL bovine serum albumin. GST is known to the expert in the field as suitable fusion partner for proteins. For this purpose, the ratio of fluorescence intensities at 520 nm (fluorescein emission excited by terbium emission) and at 495 nm (terbium emission) is determined. FIG. 1 A-E demonstrate a significant competition efficacy for the substances of this invention compound 1, compound 5, compound 4, compound 2 and compound 3.

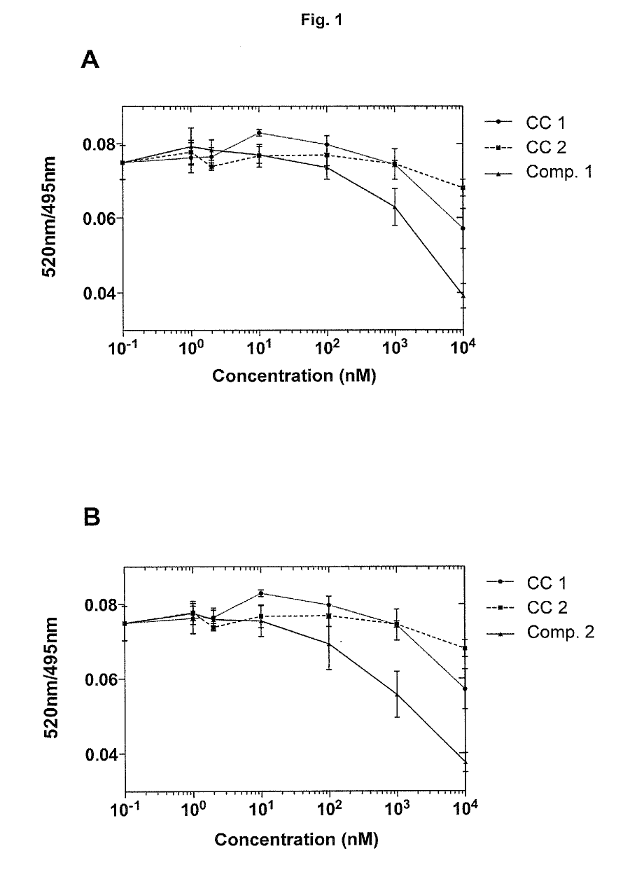

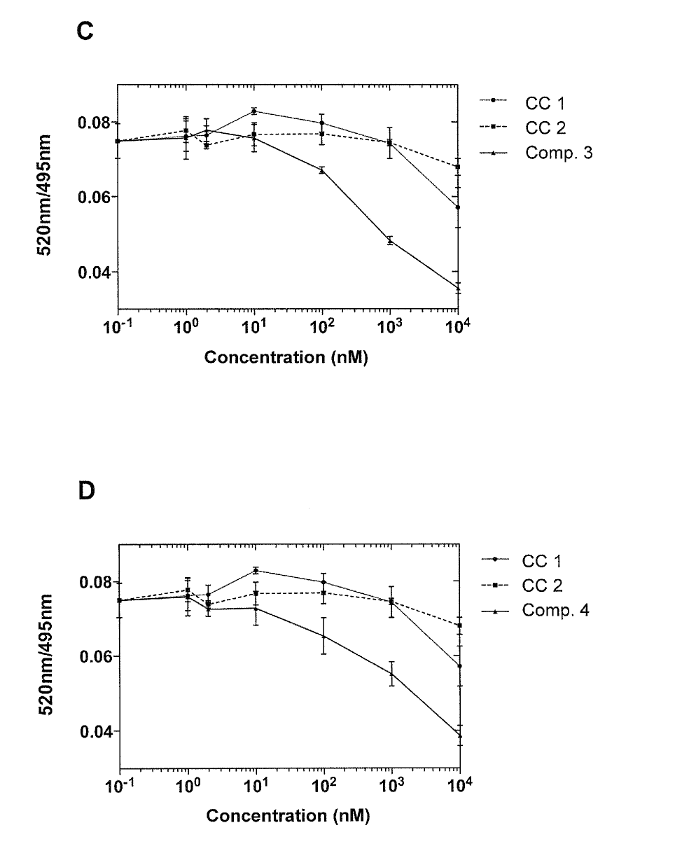

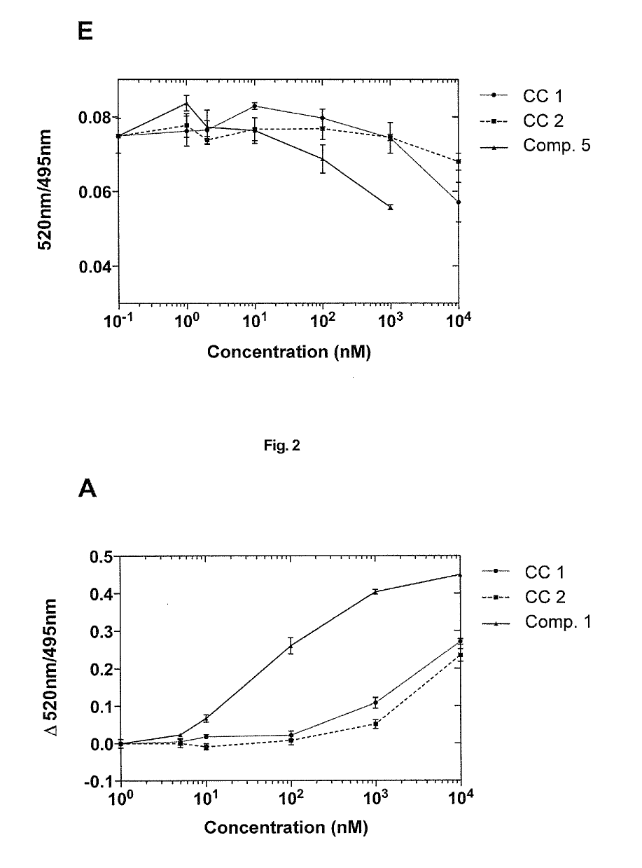

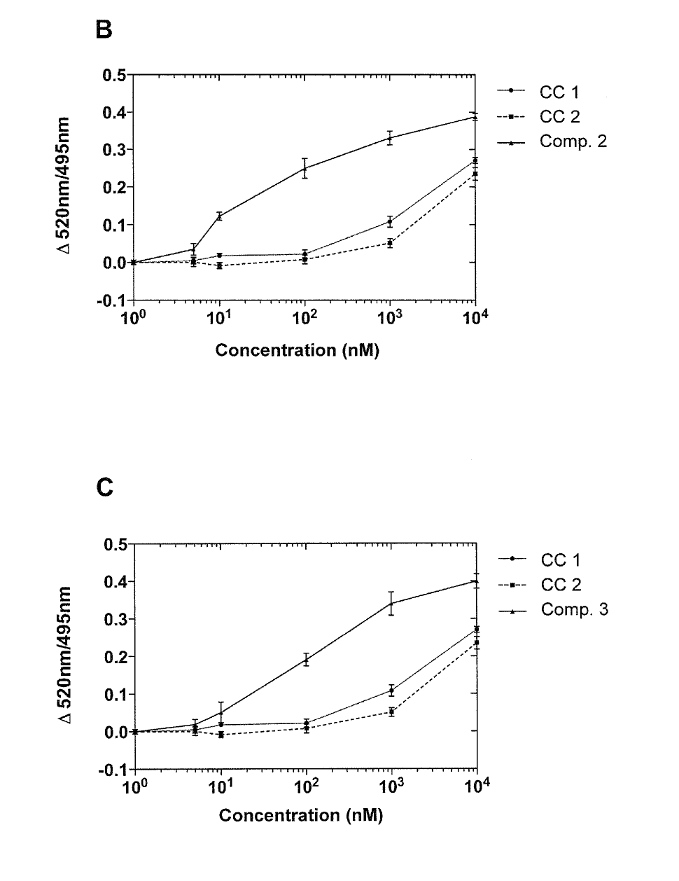

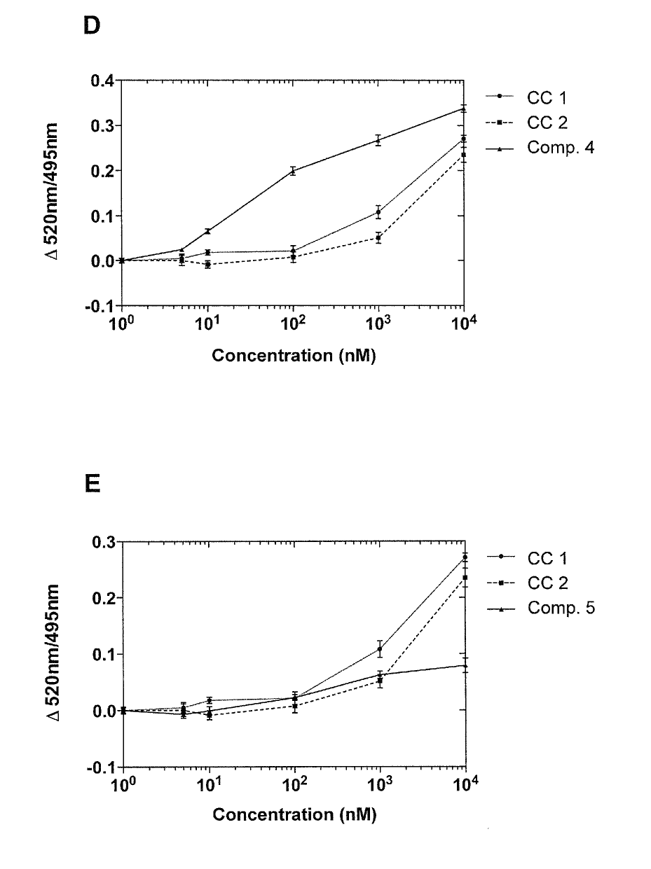

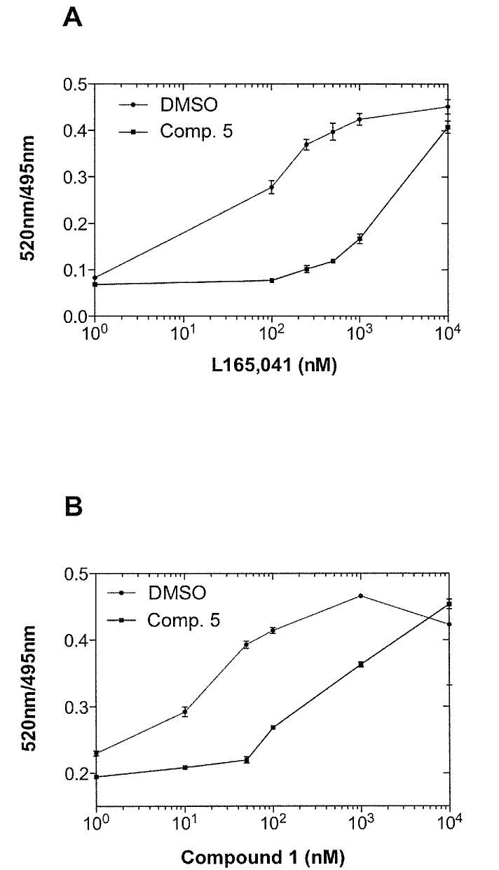

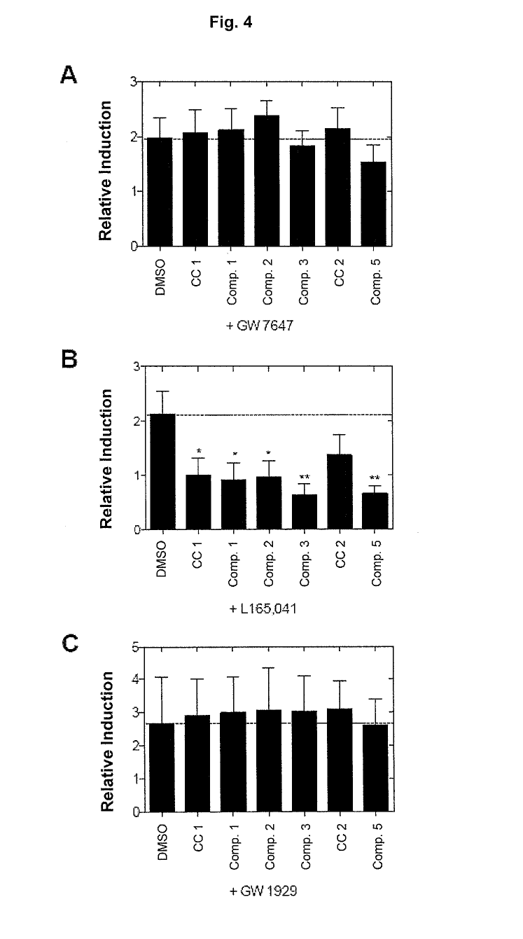

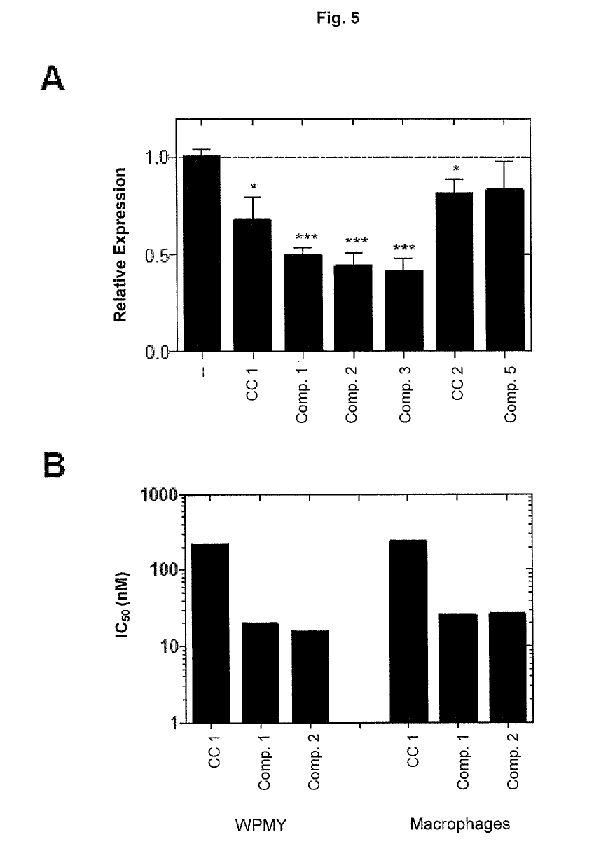

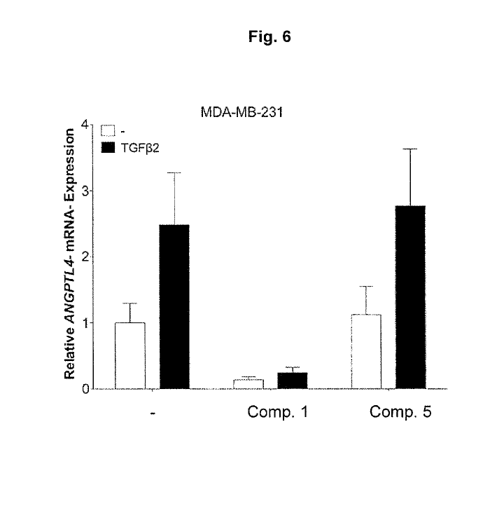

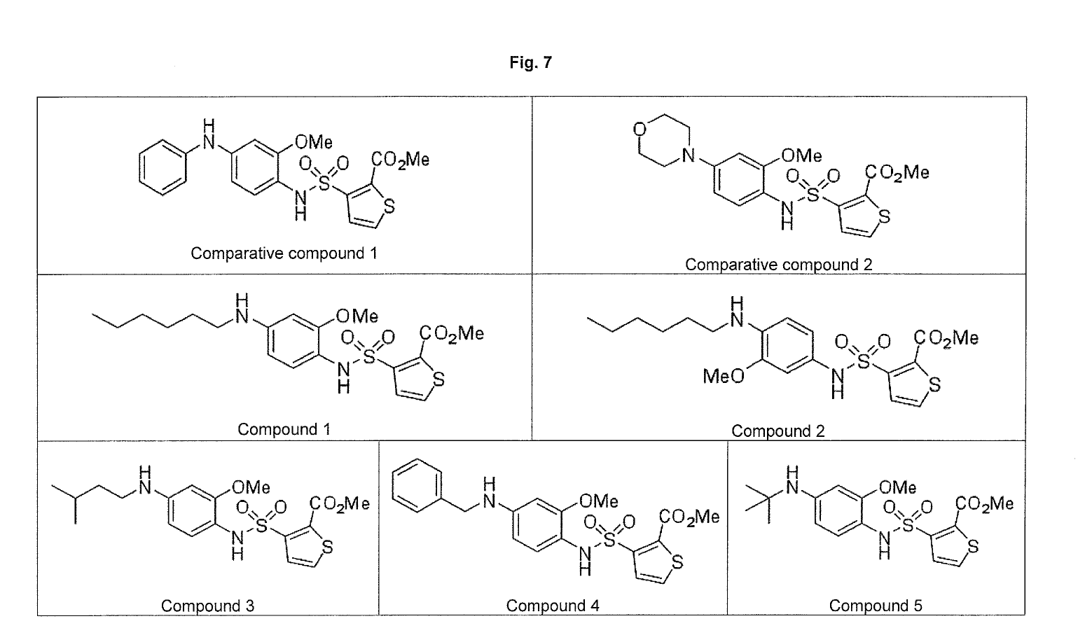

B) The ligand-induced binding of the commercially available fluorescein-labeled corepressor peptide SMRT-ID2 (HASTNMGLEAIIRKALMGKYDQW) (Invitrogen, Darmstadt, Germany) to GST-PPAR beta/delta is for example measured by TR-FRET analysis using a terbium-labeled anti-GST antibody in a VICTOR3V Multilabel Counter (WALLAC 1420; PerkinElmer Life and Analytical Sciences, Rodgau, Germany). The measurement is performed in a buffer composed of 100 mM KC, 20 mM Tris pH 7.9, 0.01% Triton X100 and 1 μg/μL bovine serum albumin. FIG. 2 A-E clearly demonstrate the interaction between SMRT-ID2 and GST-PPAR beta/delta LBD for the substances of this invention compound 1, compound 3, compound 2 and compound 4 in dependency of the respective concentration used in the assay.

C) The inhibition of the recruitment of co-activator peptide C33 known to the expert in this field mediated by PPAR beta/delta agonist L165,041 or the inhibition of the inverse agonist inventive compound 1-mediated binding of the corepressor peptide SMRT-ID2 to GST-PPAR beta/delta LBD by the substance of this invention compound 5 (1 μM) was also determined by TR-FRET measurements. Both is shown in FIGS. 3 A and B in dependency of the concentration of the agonist (A) and the inverse agonist (B), respectively.

D) The influence of substances of this invention on the agonist-induced transcriptional activity of PPAR subtypes alpha, beta/delta or gamma is determined in WPMY-1 myofibroblasts transfected transiently with a luciferase reporter plasmid (LexA system) using polyethyleneimine (PEI) according to a method known to the expert. As agonists, substances GW7647 for PPAR alpha or L165041 for PPAR beta/delta or GW1929 for PPAR gamma known to the expert are used. WPMY-1 myofibroblast cells are initially cultured for example in a 12-well plate in DMEM medium (DMEM: Dulbecco's Modified Eagle Medium) according to a method known to the expert and to a confluence of 70% to 80%, and subsequently transfected with 2.5 μg of plasmid-DNA containing the luciferase reporter plasmid, expression plasmids for the respective PPAR subtypes and the Renilla-luciferase plasmid pRL-SV40 (Promega, Mannheim, Germany), and 2.5 μl PEI (1:1000 dilution, pH 7). Four hours after transfection, the cells are treated for 48 hours with 500 nM of the substances of this invention, and 24 later with 300 nM (GW7647 or GW1929, respectively) or 500 nM (L165041) of the respective agonist at 37° C. and 5% CO₂ in an incubator. The luciferase assay is performed 48 hours after the first treatment according to the commercial method of the “dual-well-system” (pjk GmbH, Kleinblittersdorf, Germany), whereby for a normalization, not only the luciferase-, but also the Renilla activity is measured. Cells are initially lysed for this measurement, and 20 μl of the lysate is transferred into a white 96-well plate (Fisher Scientific, Hamburg, Germany). The measurement of Renilla- or luciferase-activity is performed after automatic injection of 50 μl of the respective substrate using a luminometer (Orion L Microplate Luminometer, Berthold, Düsseldorf). Mean values and standard deviation are calculated from values of three independent measurements. FIG. 4 A to C demonstrate that substances of this invention significantly reduce the induction of luciferase expression by PPAR beta/delta, but not the induction by PPAR alpha or PPAR gamma.

24.2 Influence of Substances of this Invention on the Transcription of PPAR Beta/Delta Target Genes

The influence of substances of this invention on PPAR-beta/delta regulated genes like for example ANGPTL4 which codes for the angiopoietin-like protein ANGPTL4 is assessed in different cells with a confluence of 70% to 80% in a 6-well cell culture plate. For this purpose, cultivated human myofibroblasts (WPMY-1), peritoneal mouse macrophages or cells of the human breast cancer cell line MDA-MB-231 are treated for 24 hours with 1 μM of the substances of this invention. Cells of the breast cancer cell line are in addition stimulated for 6 hours with TGF-beta2 (2 ng/ml) which is commercially available. Subsequently, RNA is isolated from the cells by procedures known to the expert and analyzed by quantitative PCR (qPCR, real time qPCR, RT-qPCR) also known to those skilled in the art. For this, cDNA is synthesized using 0.25 μg to 1 μg of RNA, oligo(dT)-primers and a commercially available cDNA synthesis kit. qPCR is then conducted in a Mx3000P RT-qPCR system (Stratagene, La Jolla, Calif., USA) according to the manufacturer's instructions with 40 cycles, an annealing temperature of 60° C., and for example human ANGPTL4 primers (fw: GATGGCTCAGTGGACTTCAACC; rv: CCCGTGATGCTATGCACCTTC) and the ribosomal I27 (fw: AAAGCCGTCATCGTGAAGAAC; rv: GCTGTCACTTTCCGGGGATAG) as normalizing gene known to the expert in this field. FIG. 5 A shows that the relative expression of the ANGPTL4 gene is reduced by up to 40% by the substances of this invention compound 1, compound 2 or compound 3 or, in the case of the comparison compound 2, by up to 10% as compared to the control (DMSO-treated cells). The respective IC₅₀ value (mean inhibitory concentration) for substances of this invention compound 1 and compound 2 is depicted in FIG. 5 B and is maximal 20 nM in WPMY-1 cells and maximal 30 nM in peritoneal mouse macrophages, respectively. FIG. 6 shows that the treatment of the breast cancer cell line MDA-MB-231 with the substance compound 1 of this invention representing an inverse agonist of PPAR-beta/delta leads to a loss of TGF-beta2-induced ANGPTL4 expression. The treatment with the substance compound 5 of this invention which represents a pure antagonist of PPAR-beta/delta however does not influence the induction of ANGPTL4 expression.

1. Compounds of the general formula (I) 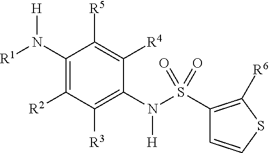
wherein
R¹ represents one of the following groups:
—CH₂—R⁷, —CH(R⁸)—R⁷, —C(R⁹)(R⁸)—R⁷, —CH₂—CH₂—R⁷, —CH(R⁹)—CH(R⁸)—R⁷, —C(R¹¹)(R¹⁰)—C(R⁹)(R⁸)—R⁷, —(CH₂)ₙ—R⁷, —R⁷, —CH₂—R³¹, —CH₂—CH₂—R³¹, —(CH₂)ₙ—R³¹,
R², R³, R⁴, R⁵ independently of one another represent the following groups:
—H, —OH, —OR¹², —OR¹³, —CF₃, —OCF₃, —F, —Cl, —Br, —I, —COR¹⁴, —COR¹⁵, —COOH, —COOR¹⁶, —COOR¹⁷, —CONH₂, —CONH(R¹⁸), —CON(R¹⁹)(R²⁰), —NH₂, —NH(R²¹), —N(R²²)(R²³), —R²⁴, —R²⁵, —OOCR²⁴, —OOCR²⁵, —R²⁶, —R²⁷, —OOC—OR²⁶, —OOC—OR²⁷, —OOC—NH₂, —OOC—NH(R²⁶), —OOC—N(R²⁶)(R²⁷), —NHCO—R²⁸;
R⁶ represents one of the following groups:
—H, —COOH, —CH₂—COOH, —COOR²⁹, —CH₂—COOR²⁹, —OH, —CH₂OH, —OR²⁹, —CH₂OR²⁹, —COR²⁹, —CONH₂, —CONH(R²⁹), —CON(R²⁹)(R³⁰), —SH, —SR²⁹, —CH₂—OOCR²⁹, —CH₂—OOC—OR²⁹, —CH₂—OOC—NH₂, —CH₂—OOC—N H (R²⁹), —CH₂—OOC—N(R²⁹)(R³⁰);
R⁷-R³° and R³⁷-R⁴¹ independently of one another represent the following groups:
—CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CH₂—CH₂-Ph, —CH₂—CH₂—CH₂-Ph, —CH═CH-Ph, —C≡C-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₃H₁₇, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, —CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —CH═CH—C(CH₃)₃, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₃H₇)═CH₂, —CH₂—C(C₂H₅)═CH—CH₃, —CH(C₂H₅)—CH═CH—CH₃, —C(C₄H₉)═CH₂, —C(C₃H₇)═CH—CH₃, —C(C₂H₅)═CH—C₂H₅, —C(C₂H₅)═C(CH₃)₂, —C[C(CH₃)₃]═CH₂, —C[CH(CH₃)(C₂H₅)]═CH₂, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH₂—CH—CH═CH—CH═CH₃, —CH═CH—CH₂—CH═CH—CH₃, —CH═CH—CH═CH—C₂H₅, —CH₂—CH═CH—C(CH₃)═CH₂, —CH₂—CH═C(CH₃)—CH═CH₂, —CH₂—C(CH₃)═CH—CH═CH₂, —CH(CH₃)—CH═CH—CH═CH₂, —CH═CH—CH₂—C(CH₃)═CH₂, —CH═CH—CH(CH₃)—CH═CH₂, —CH═C(CH₃)—CH₂—CH═CH₂, —C(CH₃)═CH—CH₂—CH═CH₂, —CH═CH—CH═C(CH₃)₂, —CH═CH—C(CH₃)═CH—CH₃, —CH═C(CH₃)—CH═CH—CH₃, —C(CH₃)═CH—CH═CH—CH₃, —CH═C(CH₃)—C(CH₃)═CH₂, —C(CH₃)═CH—C(CH₃)═CH₂, —C(CH₃)═C(CH₃)—CH═CH₂, —CH═CH—CH═CH—CH═CH₂, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —CH₂—CH(CH₃)—C≡CH, —CH(CH₃)—CH₂—C≡CH, —CH(CH₃)—C≡C—CH₃, —C₄H₈—C≡CH, —C₃H₆—C≡C—CH₃, —C₂H₄—C≡C—C₂H₅, —CH₂—C≡C—C₃H₇, —C≡C—C₄H₉, —C₂H₄—CH(CH₃)—C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —C≡C—CH₂—CH(CH₃)₂, —C≡C—C(CH₃)₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —C(CH₃)(C₂H₅)—C≡CH, —C≡C—C≡CH, —CH₂—C≡C—C≡CH, —C═C—C═C—CH₃, —CH(C≡CH)₂, —C₂H₄—C≡C—C≡CH, —CH₂—C≡C—CH₂—C≡CH, —C≡C—C₂H₄—C≡CH, —CH₂—C≡C—C≡C—CH₃, —C≡C—CH₂—C≡C—CH₃, —C≡C—C≡C—C₂H₅, —C≡C—CH(CH₃)—C≡CH, —CH(CH₃)—C≡C—C≡CH, —CH(C≡CH)—CH₂—C≡CH, —C(C≡CH)₂—CH₃, —CH₂—CH(C≡CH)₂, —CH(C≡CH)—C═C—CH₃,
R³¹ represents one of the following groups: 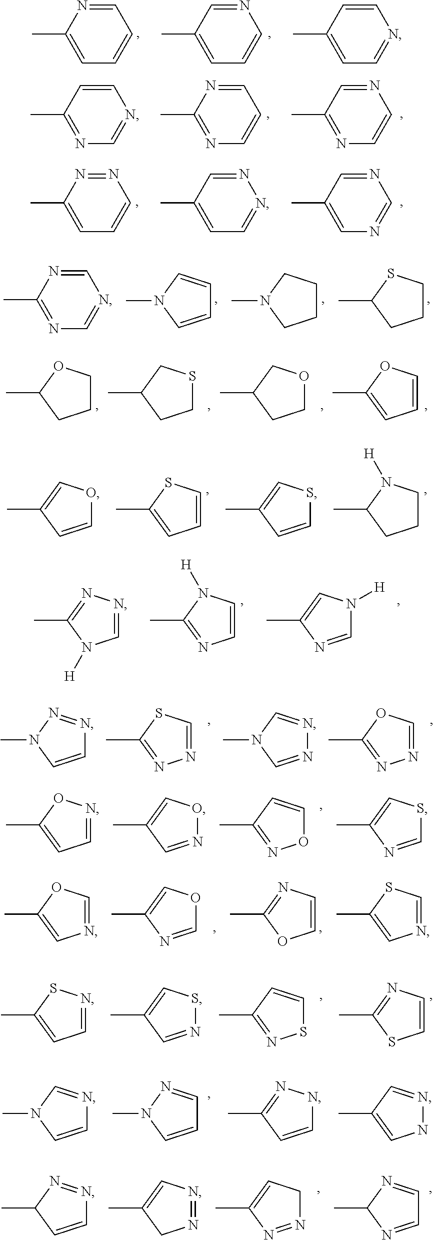 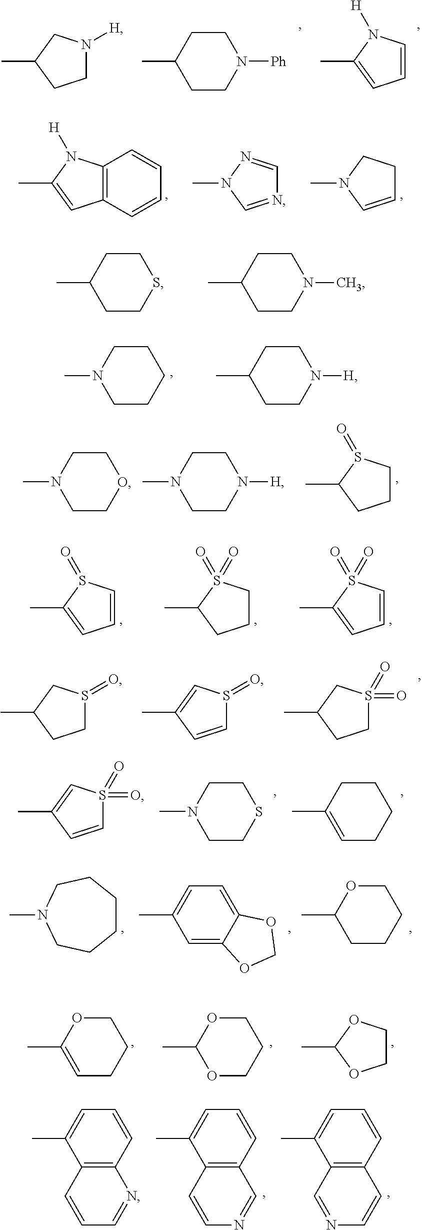 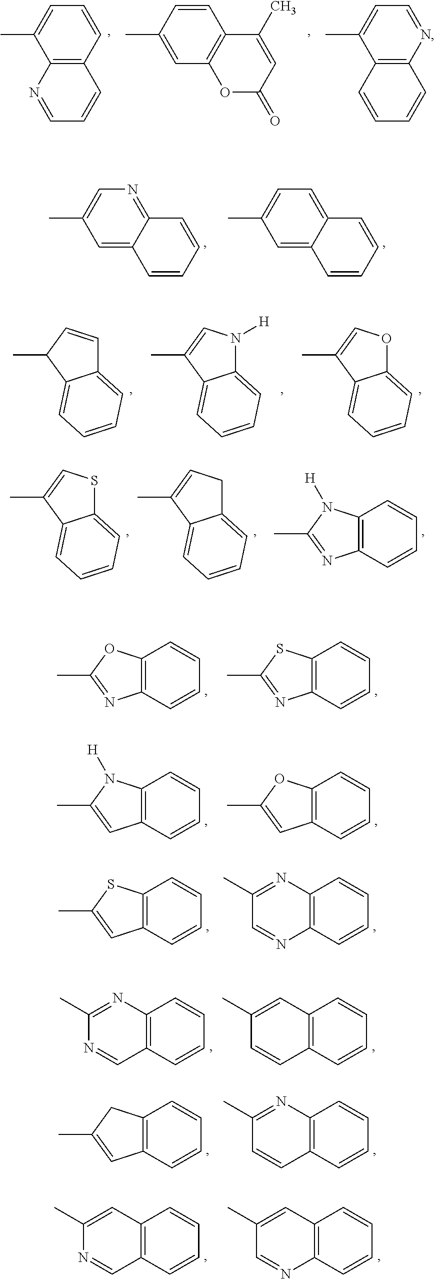 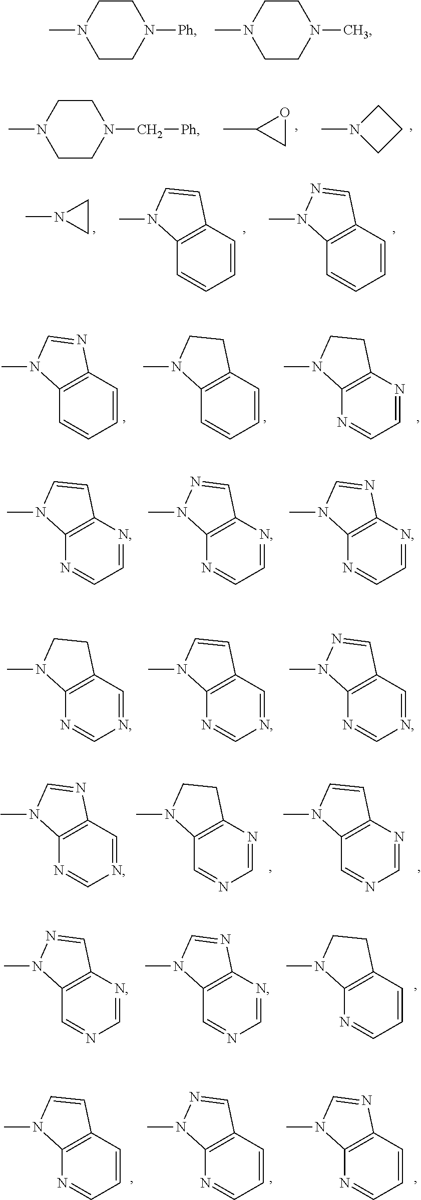 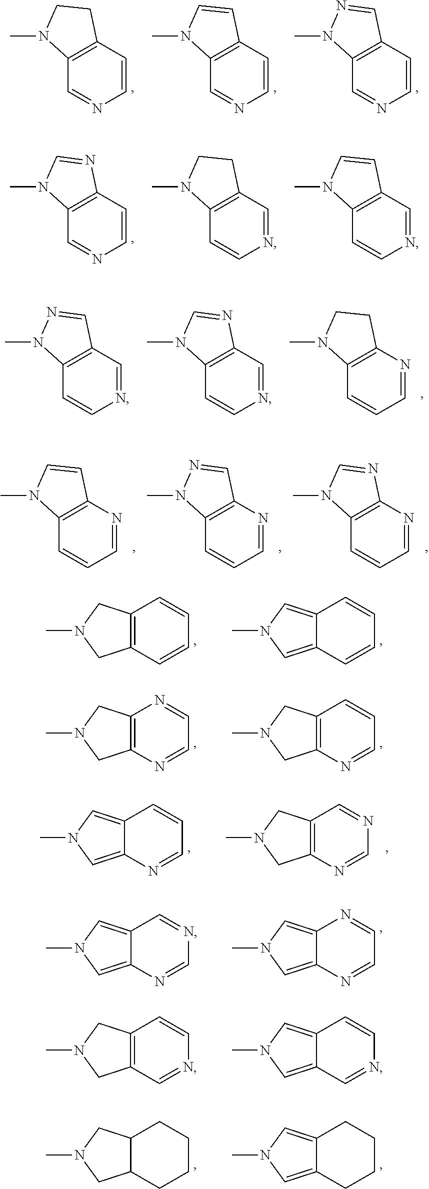 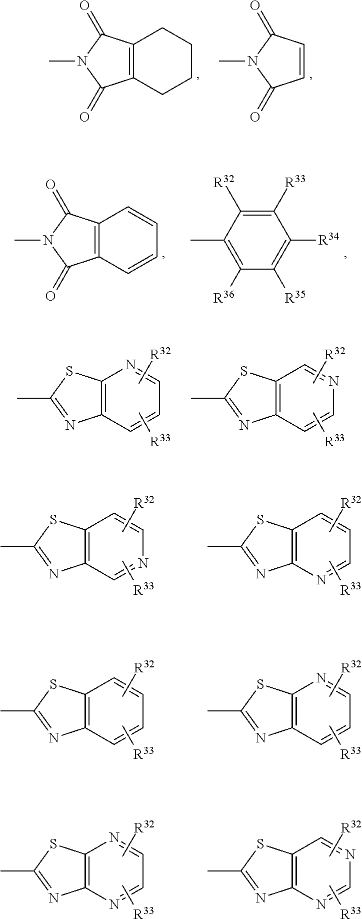
R³²-R³⁶ independently of one another represent the following groups:
—R³⁷, —R³⁸, —R³⁹, —R⁴⁰, —R⁴¹, —H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —P(O)(OH)₂, —P(O)(OCH₃)₂, —P(O)(OC₂H₅)₂, —P(O)(OCH(CH₃)₂)₂, —C(OH)[P(O)(OH)₂]₂, —Si(CH₃)₂(C(CH₃)₃), —Si(C₂H₅)₃, —Si(CH₃)₃, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CONH[CH(CH₃)₂]₂, —CONH[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —NH—C(═NH)—NH₂, —NH—C(═NH)—NHCH₃, —NH—C(═NH)—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —O—CO—NH₂, —NH—C(═NH)—N[CH(CH₃)₂]₂, —NH—C(═NH)—N[C(CH₃)₃]₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃;
n is an integer, selected from 1, 2, 3, 4 or 5;
as well as metal complexes thereof, salts, enantiomers, enantiomeric mixtures, diastereomers, diastereomeric mixtures, tautomers, hydrates, solvates and racemates of the aforementioned compounds.

2. Compounds according to claim 1, selected from the following group:
Comp. 1 3-(4-hexylamino-2-methoxyphenylsulfamoyl)-thiophene-2-carboxylic methyl ester
Comp. 2 3-(4-hexylamino-3-methoxyphenylsulfamoyl)-thiophene-2-carboxylic methyl ester
Comp. 3 3-[2-methoxy-4-(3-methylbutylamino)-phenylsulfamoyl)-thiophene-2-carboxylic methyl ester
Comp. 4 3-(4-benzylamino-2-methoxyphenylsulfamoyl)-thiophene-2-carboxylic methyl ester
Comp. 5 3-(4-tert-butylamino-2-methoxyphenylsulfamoyl)-thiophene-2-carboxylic methyl ester
Comp. 6 3-[2-methoxy-4-(propylamino)phenylsulfamoyl]thiophene-2-carboxylic methyl ester
Comp. 7 3-(4-butylamino-2-methoxyphenylsulfamoyl)-thiophene-2-carboxylic methyl ester
Comp. 8 3-[2-methoxy-4-(pentylamino)phenylsulfamoyl]thiophene-2-carboxylic methyl ester
Comp. 9 3-[2-methoxy-4-(iso-propylamino)phenylsulfamoyl]thiophene-2-carboxylic methyl ester
Comp. 10 3-(4-iso-butylamino-2-methoxyphenylsulfamoyl)-thiophene-2-carboxylic methyl ester
Comp. 11 3-(4-iso-hexylamino-2-methoxyphenylsulfamoyl)-thiophene-2-carboxylic methyl ester
Comp. 12 3-(4-iso-heptylamino-2-methoxyphenylsulfamoyl)-thiophene-2-carboxylic methyl ester
Comp. 13 3-(4-cyclohexylamino-2-methoxyphenylsulfamoyl)-thiophene-2-carboxylic methyl ester
Comp. 14 3-[2-ethoxy-4-(hexylamino)phenylsulfamoyl]thiophene-2-carboxylic methyl ester
Comp. 15 3-(4-decylamino-3-methoxyphenylsulfamoyl)-thiophene-2-carboxylic methyl ester
Comp. 16 3-(4-hexylaminophenylsulfamoyl)-thiophene-2-carboxylic methyl ester
Comp. 17 3-(4-hexylamino-2-propoxyphenylsulfamoyl)-thiophene-2-carboxylic methyl ester
Comp. 18 3-[2-butoxy-4-(hexylamino)phenylsulfamoyl]thiophene-2-carboxylic methyl ester
Comp. 19 3-(4-hexylamino-2-pentoxyphenylsulfamoyl)-thiophene-2-carboxylic methyl ester
Comp. 20 3-(4-hexylamino-2-iso-propoxyphenylsulfamoyl)-thiophene-2-carboxylic methyl ester
Comp. 21 3-(4-hexylamino-2-iso-pentoxyphenylsulfamoyl)-thiophene-2-carboxylic methyl ester
Comp. 22 3-[4-hexylamino-2-(2-phenylethoxy)phenylsulfamoyl]-thiophene-2-carboxylic methyl ester

2. Compounds according to claim 1 for a use in medicine.

3. Compounds according to claim 1 for a use as inhibitor of a receptor of the PPAR beta/delta type.

4. Utilization of compounds according to claim 1 for the treatment of inflammatory processes, inflammations, cell differentiation processes or proliferative diseases.

5. Utilization according to claim 4, whereby the proliferative diseases concerned are tumors, metastases or cancer.

6. Utilization of compounds according to claim 1 for the treatment of liver diseases.

7. Utilization of compounds according to claim 1 for the treatment of diseases of the fatty acid metabolism and the glucose metabolism in which insulin resistance is involved.

8. Pharmaceutical composition comprising at least one compound according to claim 1 and at least one pharmacologically acceptable excipient, carrier and/or at least one solvent.

9. Pharmaceutical composition according to claim 8 for the treatment of inflammatory processes, inflammations, cell differentiation processes, proliferative diseases, tumors, metastases, cancer, liver diseases as well as diseases of the fatty acid metabolism and the glucose metabolism in which insulin resistance is involved.

10. Procedure for the synthesis of compounds according to claim 1, comprising the following steps:
A1) Synthesis of a secondary para-nitroaniline from the corresponding primary para-nitroaniline by conversion with a nucleophile of the general formula R¹-LG*, wherein LG* represents a leaving group, under elimination of LG*-H which can optionally be captured with a base under formation of the corresponding salt. 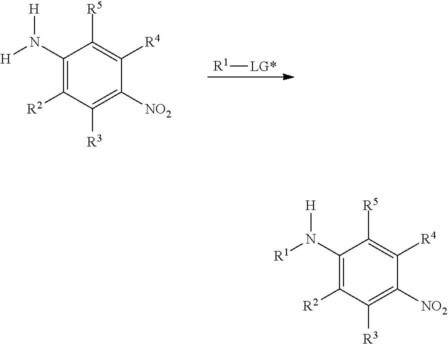
or
A2) Synthesis of a secondary para-nitroaniline from the corresponding nitrobenzene with a leaving group LG in para-position to the nitro group by conversion with a primary amine of the general formula R¹—NH₂, under elimination of LG-H which can optionally be captured with a base under formation of the corresponding salt. 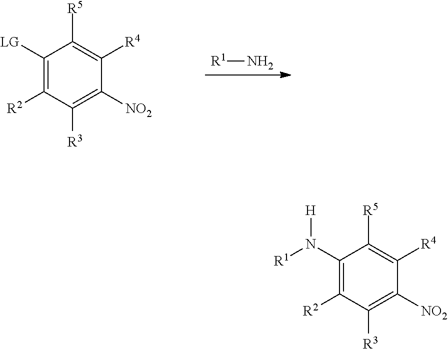
and
B) Reduction of the nitro group of the secondary para-nitroaniline obtained according to step A1) or A2) into the amino group 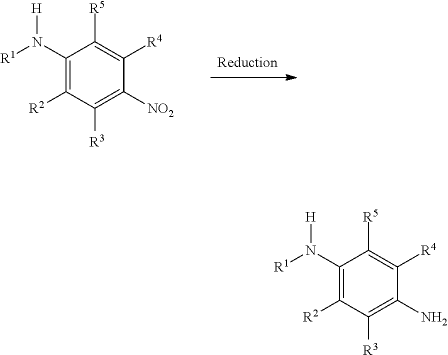
and
C) Conversion of the para-aminoaniline obtained according to step B) with a thiophene-3-sulfonylchloride under alkaline conditions into compounds of the general formula (I). [chemical structure]


SEQUENCE LISTING (5 sequences)

SEQ ID NO: 1 - DNA, 22 bp, Homo sapiens
  fw Primer angptl4 (1)..(21)
gatggctcagtggacttcaacc

SEQ ID NO: 2 - DNA, 21 bp, Homo sapiens
  rv Primer angptl4 (1)..(21)
cccgtgatgctatgcaccttc

SEQ ID NO: 3 - DNA, 21 bp, Homo sapiens
  fw Primer ribosomales l27 (1)..(21)
aaagccgtcatcgtgaagaac

SEQ ID NO: 4 - DNA, 21 bp, Homo sapiens
  rv Primer ribosomales l27 (1)..(21)
gctgtcactttccggggatag

SEQ ID NO: 5 - protein, 23 aa, Homo sapiens
  SMRT-ID2 (1)..(23)
HASTNMGLEAIIRKALMGKYDQW